(19) United States
(12) Patent Application Publication
(10) Pub. No.: US 2022/0251575 A1
(43) Pub. Date: Aug. 11, 2022

(54) Modulation of Engineered Immune Cell Receptor Translation Using Noncoding Sequence Elements

(71) Applicant: The Regents of the University of California, Oakland, CA (US)

(72) Inventors: James H. D. Cate, Berkeley, CA (US); Dasmanthie De Silva, Berkeley, CA (US)

(73) Assignee: The Regents of the University of California, Oakland, CA (US)

(21) Appl. No.: 17/726,517

(22) Filed: Apr. 21, 2022

Related U.S. Application Data
(63) Continuation of application No. PCT/US20/56942, filed on Oct. 22, 2020.
(60) Provisional application No. 62/924,680, filed on Oct. 22, 2019.
(60) Provisional application No. 62/952,185, filed on Dec. 20, 2019.

(51) Int. Cl. C12N 15/62; C12N 15/11; C12N 9/22; C12N 5/0783; C12N 15/85; A61K 35/17

(52) U.S. Cl. CPC C12N 15/625; C12N 15/11; C12N 9/22; C12N 5/0636; C12N 15/85; C12N 2501/2302; C12N 2310/20; C12N 2800/80; C12N 2830/85; C12N 2800/107; A61K 35/17

(57) ABSTRACT

Engineered immune cell receptor translation is modulated using heterologous noncoding sequence elements, such as modified eukaryotic initiation factor 3 (eIF3) responsive sites.

This invention was made with government support under Grant Numbers GM065050 and GM102706 awarded by the National Institutes of Health. The government has certain rights in the invention.

Reference to a Sequence Listing. A Sequence Listing in text format is incorporated by reference into the specification. The name of the text file containing the Sequence Listing is B20-053_ST25.txt. The text file is 6,425 bytes and was created on Oct. 10, 2020.

### INTRODUCTION

Cancer immunotherapy has reached a watershed moment, with its benefits in treating cancer starting to be realized. CAR T-cell therapy, in which patient blood T cells are genetically engineered to recognize a specific tumor-associated antigen, has proven powerful in treating some cancers. For example, CAR Ts demonstrate exceptional clinical efficacy against B cell malignancies, and two therapies, Kymriah™ (tisagenlecleucel, Novartis) and Yescarta™ (axicabtagene ciloleucel, Kite/Gilead), were recently approved by the FDA. However, the uncontrolled intensity of the immune response-termed a “cytokine storm”, also known as “cytokine release syndrome” or CRS, observed in over 50% of patients that can be very dangerous or even lethal to patients (Jensen, 2014). CRS is characterized by high elevations of cytokines including INF-γ, granulocyte-macrophage colony-stimulating factor (GMCSF), IL-10, and IL-6. These cytokine elevations result in a plethora of clinical symptoms including fever, hypotension, organ dysfunction, respiratory failure and coagulopathy. Additionally, neurotoxicity often presents even after the initial symptoms of CRS have subsided. The pathogenesis of CRS and associated neurotoxicity is poorly understood and further understanding of the mechanism would be useful for the successful translation of CAR-T therapy. In the meanwhile, disrupting the pathogenesis of CRS by reducing the level of cytokine genes available for expression is one way to mitigate the condition. In addition, other cell-based therapies are now in development that also use immune cells to target cancers (engineered NK cells), or to treat autoimmune diseases or elicit organ transplant tolerance (engineered regulatory T cells) (Ferreira, 2019).

Activation of immune cells requires a global increase in cellular protein synthesis, which is largely achieved by an increase in translation initiation. This suggests that the regulation of translation initiation plays a major role during immune cell activation and function. However, the precise molecular events in translation initiation that occur during immune cell activation and function are not understood. It has been shown that a central component of the translational machinery—eukaryotic initiation factor 3 (eIF3), a multi-subunit complex comprised of subunits EIF3A through EIF3M—is mostly turned off in quiescent T cells, but is dramatically activated for translation initiation upon T cell activation. Stimulation of eIF3 activity in activated T cells requires the association of one of its subunits, EIF3J (or eIF3j) (Miyamoto, 2005). Furthermore, activation of T cells leads to tyrosine phosphorylation of eIF3 subunits (Matsumoto, 2009). However, the exact role of eIF3 in T cell activation remains unexplored.

Several lines of evidence indicate eIF3 serves specialized roles in cellular translation, by recognizing specific RNA structures in the 5′-untranslated regions (5′-UTRs) of target mRNAs (Lee et al (2015) Nature 522:111-114), by binding the 7-methyl-guanosine (m7G) cap (Lee et al (2016) Nature 536:96-99) or through interactions with N-6-methyl-adenosine (m6A) post-transcriptional modifications in mRNAs (Meyer et al (2015) Cell 163:999-1010). Binding to these cis-regulatory elements in mRNA can lead to translation activation or repression, depending on RNA sequence and structural context (Lee, 2015 ibid; Meyer, 2017, ibid; de la Parra et al (2018) Nat Commun 9:3068). These functions for eIF3 can aid cell proliferation, or allow cells to rapidly adapt to stress such as heat shock. Additionally, eIF3 plays an important role in the development of specific tissues.

Although eIF3 is a general translation initiation factor, we recently discovered that eIF3 has built-in specificity and directly controls the translation of specific mRNAs that encode key regulatory proteins involved in, for example, cell cycling, differentiation and apoptosis (Lee, 2015; Lee, 2016). Surprisingly, eIF3 can either activate or repress the translation of these mRNAs by binding to the 5′ untranslated region (5′-UTR) of the mRNAs,. This finding identified a fundamentally new role for eIF3 in regulating special programs of gene expression and the binding of eIF3 to these specific mRNAs could be involved in carcinogenesis. However, these results were obtained from human kidney-derived cells, leaving open the question of how eIF3 functions in T cell activation.

### SUMMARY OF THE INVENTION

It would be beneficial to control the expression of a heterologous gene (for example, a nucleic acid encoding an engineered immune cell receptor or other gene of interest) for use in cell-based cancer immunotherapy, known as adoptive cell therapy (ACT), or other cell-based therapies using engineered regulatory T cells (engineered Tregs) to treat immune dysfunction such as autoimmunity or organ transplant rejection. In some types of these therapies, immune cells such as T cells or natural killer (NK) cells are genetically modified to express an engineered cell surface receptor such that when these modified immune cells are given to a subject, the cells are directed to tumor cells or specific tissues expressing a target ligand recognized by the receptor, thereby leading to tumor cell destruction (ACT) or moderated immune reaction (engineered Tregs). In other types of cell-based therapies, engineered stem cells comprising genes encoding engineered immune cell receptors or other therapeutic polypeptides are introduced in a subject. The introduced stem cells will differentiate into targeted immune cells and will provide a long-term reservoir of these therapeutic cells in the body. The engineered receptors can include, among others, T cell receptors (TCRs) and chimeric antigen receptors (CARs) as reviewed in the literature (Sadelain, 2017; Zhang, 2018; Souza-Fonseca-Guimaraes, 2019; Lee, 2019; Paucek, 2019; Ferreira, 2019). However, it has been found that subjects treated with ACT can suffer from severe toxic side effects including cytokine release syndrome (CRS), graft-versus-host disease (GvHD), and neurotoxicity, in some cases leading to death of the patient. These toxicities may arise due to overactivation of the engineered immune cells used in ACT such as CAR T-cells, due to dysregulated signaling by the engineered cell surface receptor. Conversely, overactive immune cells can become exhausted and lose efficacy over time. Present attempts to regulate CAR expression through modifications such as altering receptor specificity and modification of intracellular signaling domains or through the introduction of a “safety switch” to shut off receptor expression (Dwivedi et al (2018) Front Immunol 9:3180) do not take into account the possibility of regulation at the level of protein synthesis. It would therefore be useful to be able to tune the activity of stem and immune cells engineered for ACT, by either increasing or decreasing the protein synthesis of the engineered immune cell surface receptor, i.e. the engineered TCR or CAR.

The use of engineered TCRs or CARs typically involves genetically modifying stem, T or NK cells with a DNA sequence encoding the engineered TCR or CAR. Many such methods have been described in the literature to introduce a gene encoding an engineered receptor involving, for example, retroviral vectors (Imai, 2005; Liu, 2018), transposons (Kabriaei, 2016), and more recently the use of CRISPR-Cas9 (Eyquem, 2017; Roth, 2018). Using such methods, it is possible to stably introduce DNA sequences into primary T cells, NK cells and induced pluripotent stem cells (iPSCs) that are subsequently differentiated into T cells (Nishimura, 2019), regulatory T cells (Hague, 2016) or NK cells (Bernareggi, 2019). These modified cells can then be expanded and used for ACT, organ transplantation, or treatment of autoimmune diseases.

Thus, provided herein are methods and compositions for regulation of the translation of a heterologous polypeptide in an immune or stem cell. In some aspects, the heterologous protein is an engineered receptor (for example a CAR or TCR) or other beneficial polypeptide for use in cell therapy. In some aspects, the mRNAs encoding the heterologous protein comprise one or more non-coding sequences in the 3′ untranslated region (UTR). In some aspects, the 3′-UTR sequences are sensitive to regulation by translation initiation factor eIF3 to modulate the strength and time duration of TCR or CAR protein synthesis.

In some aspects, the gene encoding the exogenous polypeptide comprises a heterologous 3′ UTR sequence that can be regulated at a translational level by eIF3. In some aspects, the gene comprises one or more (for example, 1, 2, 3, 4, or 5) 3′ UTR sequences that can be regulated by eIF3. In some aspects, the gene comprises a mutated 3′UTR sequence that can be regulated by eIF3. In some of any embodiments, the provided polynucleotide encoding the gene and the 3′UTR sequence is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some of any embodiments, the polynucleotide is between at or about 2500 and at or about 5000 nucleotides, at or about 3500 and at or about 4500 nucleotides, or at or about 3750 nucleotides and at or about 4250 nucleotides in length.

In certain aspects, provided herein is a genetically modified cell comprising a heterologous protein. Populations of these genetically modified cells are also provided as are descendants from such cells. The heterologous protein may comprise an engineered receptor, for example a chimeric antibody receptor (CAR), T cell receptor (TCR) or antibody coupled T cell receptor (ACTR). In some aspects, the gene encoding the heterologous receptor also encodes one or more heterologous 3′ UTRs. In some aspects, the 3′UTR is be regulated by eIF3.

In some aspects, the gene encoding the heterologous polypeptide is inserted into the genome of the cell. In some aspects, the gene is inserted into the genome through the use of an engineered nuclease or nuclease system. In some aspects, the engineered nuclease or nuclease system is a CRISPR/Cas system, a zinc finger nuclease, a transcription activator-like effector nuclease (TALEN), a MegaTAL or a meganuclease. In some aspects, an engineered nuclease or a nuclease system is provided to the cell before or after the gene sequence for insertion is provided. In some aspects, the nuclease or nuclease system is provided to the cell at the same time as the gene sequence for insertion. In some aspects the gene is inserted into a safe harbor locus.

In some aspects, the gene encoding the heterologous polypeptide is inserted randomly in the genome (for example, using a lentiviral delivery system).

In some aspects, the gene encoding the heterologous polypeptide is maintained episomally in the cell and is not integrated into the cell chromosomes. In some aspects, the gene is maintained in a plasmid, a mini-plasmid or a non-integrating viral vector (for example, using an integrase defective lentiviral vector (IDLY) or an adeno-associated viral vector (AAV)).

In some aspects, the gene encoding the heterologous polypeptide is transcriptionally regulated by an endogenous promoter, wherein in other aspects, the gene is transcriptionally regulated by an exogenous promoter. In some aspects, the gene is inserted downstream of an endogenous promoter in the cell genome. In some aspects, the endogenous promoter is constitutive while in other aspects, the endogenous promoter is inducible. In some aspects where the gene encoding the heterologous polypeptide is maintained on an episome, the gene is transcriptionally regulated by a promoter that may be constitutive or inducible.

In some aspects, provided herein is a cell comprising a gene encoding the heterologous polypeptide wherein the gene also comprises sequences encoding one or more heterologous 3′UTR sequences regulated by eIF3. In some aspects, the cell is generated by contacting the cell with a gene encoding the heterologous polypeptide comprising the one or more heterologous 3′ UTR sequences regulated by eIF3. In some aspects the contacting is done with viral delivery, lipid nanoparticle (LNP) delivery or with naked DNA. In some aspects the viral delivery is carried out using an AAV vector such as an AAV vector selected from among AAV1, AAV2, AAV3, AAV4, AAV5, AAV6, AAV7 or AAV8 vector. In some aspects the viral delivery is carried out using a retroviral or lentiviral vector.

In certain aspects, the cell is an immune cell (for example, a T regulatory cell, a T effector cell or an NK cell) while in other aspects, the cell is a stem cell (for example a hematopoietic stem cell). In some aspects, the cell is patient-derived, for example a CD4+ (hematopoietic) stem cell (e.g. mobilized in a subject from the bone marrow into the peripheral blood via granulocyte colony-stimulating factor (GCSF) or plerixafor (for example Mozobil)) that can be isolated and modified to comprise the gene encoding the heterologous polypeptide. In some aspects, the cell is derived from a healthy human volunteer. In some aspects, the stem or immune cells may used for cell therapy, for example, for a T cell transplant using mature modified T cells. In some aspects, the cell as disclosed herein comprises additional modifications. In some aspects, the cell comprises modifications of immunomodulatory factors (e.g. PD1, CTLA-4 etc) and/or self antigens (e.g. MHC).

In some aspects, the gene encoding the heterologous polypeptide encodes a receptor that is useful for cell therapy. In some aspects the receptor is a CAR or TCR wherein the receptor is specific for a tumor antigen or an antigen associated with an infectious agent (for example, HIV or Aspergillus, see Parida et al (2015) Clin Infect Dis 61(supp13): S217-S224). In some aspects the CAR is bi-specific. In some aspects, the TCR is derived from a tumor infiltrating lymphocyte (TIL).

In some aspects the antigen is a tumor antigen such as an antigen selected from among glioma-associated antigen, b-human chorionic gonadotropin, alpha fetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp1OO, MAGE-A1, MAGE-A2, MAGE-A3, MAGE- A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), b-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

In some aspects, the antigen is a viral antigen, such as an antigen selected from a viral antigen from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV). In some aspects, the antigen is an antigen from an HPV selected from among HPV-16, HPV-18, HPV-31, HPV-33 and HPV-35, such as an HPV-16 antigen that is an HPV-16 E6 or HPV-16 E7 antigen. In some aspects, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In some aspects, the viral antigen is an HTLV-antigen that is TAX. In some aspects, the viral antigen is an HBV antigen that is a hepatitis B core antigen or a hepatitis B envelope antigen.

In some aspects, a composition of modified cells of the invention is provided. In some aspects, the composition comprises patient-derived (autologous) modified cells while in other aspects, the composition comprises healthy volunteer-derived (allogenic) modified cells. In some aspects, 10%, 20%, 30%, 40%, 50%, 60%, 70%, 80%, 90% or 100% of the cells of the composition comprise the gene encoding the heterologous polypeptide and 3′ UTR sequence are provided.

Also provided herein are methods, for example, methods for producing a modified cell wherein translation of a heterologous polypeptide that is not normally regulated at a translational level is regulated by eIF3. In some aspects, provided herein is a method of producing an engineered T, NK or stem cell containing a heterologous polypeptide that is encoded by a gene comprising one or more 3′ UTRs that are regulated by eIF3, the method including: (a) introducing into the T, NK or stem cell one or more engineered nuclease or nuclease system that is/are capable of introducing a break in the genome of the cell and (b) introducing the gene encoding the heterologous polypeptide wherein the gene comprises a heterologous 3′ UTR that is regulated by eIF3; wherein the gene is inserted into the break via homology directed repair (HDR) or end capture via non-homologous end joining (NHEJ), thereby producing a modified cell comprising a gene encoding the heterologous polypeptide wherein translation of the polypeptide is regulated by eIF3. In some aspects, provided herein is a method of producing an engineered T, NK or stem cell containing a heterologous polypeptide that is encoded by a gene comprising one or more heterologous 3′UTRs that are regulated by eIF3, the method including: (a) introducing into the T, NK or stem cell an episomal vector comprising the gene encoding the heterologous polypeptide that comprises one or more heterologous 3′ UTRs that are regulated by eIF3.

In some aspects, provided herein are methods of producing an engineered T, NK or stem cell comprising a CAR wherein translation of the CAR that is not normally regulated at a translational level is regulated by eIF3. In some aspects, provided herein are methods of producing an engineered T, NK or stem cell comprising an engineered TCR that is not normally regulated at a translational level is regulated by eIF3.

In some aspects, provided herein is a use of any of the engineered cells, populations of engineered cells, compositions of engineered cells or cells derived from any of the engineered cells in the treatment of a disease or disorder for example by administering the cells to a subject in need thereof.

In some aspects, provided herein is a use of any of the engineered cells, populations of engineered cells, compositions of engineered cells or cells derived from an of the engineered cells in the manufacture of a medicament for treating a disease or disorder for example by administering the cells to a subject in need thereof.

In some aspects, provided herein is an engineered cell, population of engineered cells, composition of engineered cells or cells derived from such engineered cells for use in the treatment of a disease or disorder for example by administering the cells to a subject in need thereof.

A kit, comprising the nucleic acids, and/or cells of the invention is also provided. The kit may comprise nucleic acids encoding the nucleases or nuclease systems, or aliquots of the nuclease proteins or RNPs comprising the nuclease proteins, viral delivery vectors as well as nucleic acids encoding the engineered heterologous polypeptides and instructions for performing the methods and uses of the invention and the like.

### BRIEF DESCRIPTION OF THE DRAWINGS

FIGS. 1A through 1F: PAR-CLIP analysis of eIF3 interactions with RNAs in Jurkat cells. (a) Schematic of Jurkat cell treatment with 4-thiouridine, activation, and cell harvesting for PAR-CLIP analysis. (b) Crosslinking of eIF3 subunits EIF3A, EIF3B and EIF3D across the entire TCRA and TCRB mRNAs. 5′-UTR, coding sequence (CDS), and 3′-UTR elements for the mapped TCR genes in Jurkat cells are shown. Counts, PAR-CLIP reads from deep sequencing library analysis (Corcoran, 2011). (c) Phosphorimage of SDS polyacrylamide gel resolving 5′ ³²P-labeled RNAs crosslinked to eIF3 subunits in activated and non-activated Jurkat cells. (d) Composition of eIF3 in I+PMA activated and non-activated Jurkat cells after anti-EIF3B immunoprecipitation (IP), identified by mass spectrometry. Shown is an SDS polyacrylamide gel stained with Coomassie Brilliant Blue. (e) Composition of eIF3 in activated Jurkat cells determined by western blot after anti-EIF3B IP. IN: input; FT: flow-through from anti-EIF3B IP beads; ELU: elution of eIF3 from anti-EIF3B IP beads. Anti-EIF4G1 and anti-RPS19 western blots confirm stringency of bead wash steps. (f) FISH analysis of TCRA and TCRB mRNAs (yellow and magenta, respectively) and c, stress granules (on the right) marked by the location of G3BP1 (cyangreen) or d, P bodies (on the left) marked by the location of DCP1 (cyangreen), in activated Jurkat cells. Graphs to the right of the images indicate Pearson's correlation coefficients (PCCs) of TCRA and TCRB mRNA with each other or with stress granules or P bodies (n=5, P<0.008, for PCC values of cells relative to bead colocalization, across all the channels tested, using the Wilcoxon rank-sum test).

FIGS. 2A and 2B: TCRA protein levels in Jurkat cells with eIF3 3′-UTR PAR-CLIP sites deleted. (a) eIF3-mRNA PAR-CLIP sites in TCRA and TCRB genes, with sites in the 3′-UTRs deleted as marked. In TCRA, sgRNA1 and sgRNA2 target hg38 genomic locations chr14: 22,551,700 and chr14: 22,552,073, respectively. In TCRB, sgRNA1 and sgRNA2 target hg38 genomic locations chr7: 142,802,561 and chr7: 142,802,695. (b) Western blots measuring TCRA protein levels as a function of time after anti-CD3/anti-CD28 activation. Cell lines are indicated by transcript structure above the western blots. HSP90 was used as a loading control.

FIGS. 3A through 3C: TCRA and TCRB mRNA levels in Jurkat cells with eIF3 3′-UTR PAR-CLIP sites deleted. (a) Fold changes in mRNA levels in both WT cells and in cells with either the TCRA or TCRB 3′-UTR/eIF3 PAR-CLIP sites deleted, as determined by qRT-PCR at different time points after anti-CD3/anti-CD28 activation. (b) Immunoprecipitation of eIF3 using an anti-EIF3B antibody (Lee, 2015), followed by qRT-PCR to quantify the amount of TCRA or TCRB mRNA bound to eIF3. Jurkat cells with or without the eIF3 PAR-CLIP site in the 3′-UTR were analyzed after activation with anti-CD3/anti-CD28 antibodies for 5 hours and 8 hours. Percent input (TCRA or TCRB on the left, IL2 on the right), percentage of mRNA relative to total mRNA isolated from the cells, prior to EIF3B immunoprecipitation. (c) Percent mRNA bound to anti-EIF3B beads relative to total mRNA isolated from the cells (TCRA or TCRB on the left, IL2 on the right). (n=3, with mean and standard deviations shown). For TCRA and TCRB mRNAs, P<0.0001 relative to WT. P values for IL2 mRNAs for ΔPAR relative to WT were not significant using two-way ANOVA.

FIGS. 4A through 4D: Role of the eIF3 PAR-CLIP sites in the 3′-UTRs of TCRA and TCRB, measured by nanoluciferase reporters. (a) Schematic of the nanoluciferase reporters stably expressed in Jurkat cells. WT, intact 3′-UTR from either TCRA or TCRB mRNA; ΔPAR, 3′-UTR of TCRA or TCRB with the eIF3 PAR-CLIP site deleted (nucleotides 102-338 in the 3′-UTR of TCRA mRNA or nucleotides 16-161 in the 3′-UTR of TCRB mRNA); R*PAR, reversed PAR-CLIP sequence. (b) Luciferase activity in anti-CD3/anti-CD28 activated Jurkat cells, relative to non-activated controls. (c) Fold changes in mRNA levels in cells stably expressing the nanoluciferase reporters, as determined by qRT-PCR. (d) Immunoprecipitation of eIF3 using an anti-EIF3B antibody (Lee, 2015), followed by qRT-PCR to quantify the amount of nanoluciferase mRNA bound to eIF3. Results are from biological triplicates, with standard deviation shown.

FIGS. 5A and 5B: Phenotypic analysis of Jurkat cells with the eIF3 PAR-CLIP sites in either the TCRA or TCRB 3′-UTR deleted. (a) Flow cytometric analysis of cells expressing T cell activation markers CD69 (early activation marker) and CD25 (mid-activation marker) in WT, TCRA ΔPAR or TCRB ΔPAR cells at different time points after stimulation with anti-CD3/anti-CD28 antibodies. (b) Quantification of secreted IL2 from WT, TCRA ΔPAR or TCRB ΔPAR cells at different time points after stimulation with anti-CD3/anti-CD28 antibodies using ELISA.

FIG. 6: Total genomic DNA extracted from WT, TCRA ΔPAR, TCRB ΔPAR, cells nucleofected with scrambled gRNA and cells not nucleofected were analyzed by PCR to measure the editing efficiency. The PCR primers were designed 400-500 base pairs (bp) from either side of the editing site. TCRA ΔPAR cells (top) produced a 1283 bp PCR product compared to 1676 bp in WT, and TCRB ΔPAR cells (bottom) produced a 1022 bp PCR product compared to 1190 bp in WT.

FIGS. 7A-7E: TCRA protein levels in primary human T cells with eIF3 3′-UTR PAR-CLIP sites deleted. (a) Western blots measuring TCRA protein levels as a function of time after anti-CD3/anti-CD28 activation. Cell lines are labeled on the left. HSP90 was used as a loading control. (b) Percentage of cells expressing TCR on the cell surface measured by flow cytometric analysis. On the left, the TCR expressing cells were quantified and presented in a bar graph depicting all the cell lines used: 1gB, TCRB gRNA 1; 2gB, TCRB gRNA 2; 1+2 gB, TCRB gRNA 1+2; 1gA, TCRA gRNA 1; 2gA, TCRA gRNA 2; 1+2 gA, TCRA gRNA 1+2; KO, TCR gRNA targeting the CDS of TCRA; N, Non-nucleofected cells; S, Scrambled gRNA. On the right, TCR expressing cells presented in density plots for cell lines 1+2 gB, 1+2 gA, KO and S. (c) Fluorescent confocal airyscan microscopy of TCRA protein in Primary human T cells. TCR protein is in yellow in the right panels. The left panels have the merged image with TCRA protein in yellow, TCRA RNA in pink and DAPI-stained nucleus. (d) The number of WT, TCRA ΔPAR, TCRB ΔPAR cells expressing an immune synapse measured using an epifluorescence microscope 1 h, 3 h and 5 h after activation with anti-CD3/anti-CD28 antibodies. (e) Percent cells expressing TCR on the cell surface after I+PMA activation for 3 hours (n=2 donors, with mean and standard deviation shown), reported in bar graph form. P<0.0001 for TCRA ΔPAR or TCRB ΔPAR relative to SC, using one-way ANOVA.

FIGS. 8A-8F: Phenotypic analysis of primary human T cells with the eIF3 PAR-CLIP sites in either the TCRA or TCRB 3′-UTR deleted. (a) Gating strategy used for flow cytometric analysis of primary human T cells expressing T cell activation markers CD69 and CD25 after activation with anti-CD3/anti-CD28 antibodies. (b) and (d) Flow cytometric analysis of CD8+ and CD4+ T cells expressing T cell activation markers CD69 (early activation marker) and CD25 (mid-activation marker) in the following cells at different time points after stimulation with anti-CD3/anti-CD28 antibodies: 1gB, TCRB gRNA 1; 2gB, TCRB gRNA 2; 1+2 gB, TCRB gRNA 1+2; 1gA, TCRA gRNA 1; 2gA, TCRA gRNA 2; 1+2 gA, TCRA gRNA 1+2; KO, TCR gRNA targeting the CDS of TCRA; N, Non-nucleofected cells; S, Scrambled gRNA. (c) and (e) Density plots depicting the percentage of WT, TCRA ΔPAR, TCRB ΔPAR and KO (ΔTCR) cells expressing T cell activation markers CD69 and CD25 after activation with anti-CD3/anti-CD28 antibodies at different time points. (f) Quantification of secreted IL2 and IFNγ from 1gB, 2gB, 1+2 gB, 2gA, 1+2 gA, KO, N and S cells at different time points after stimulation with anti-CD3/anti-CD28 antibodies using an ELISA.

### DETAILED DESCRIPTION

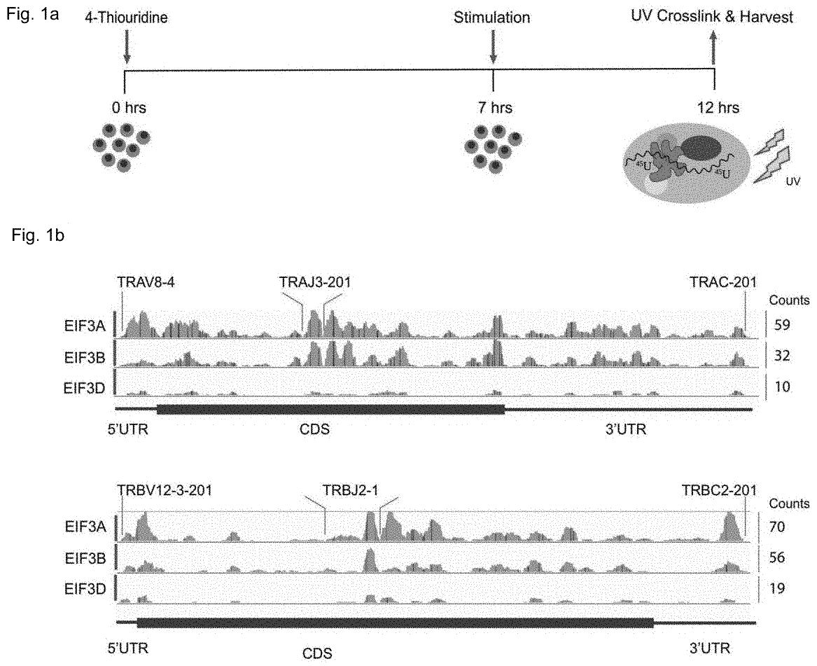

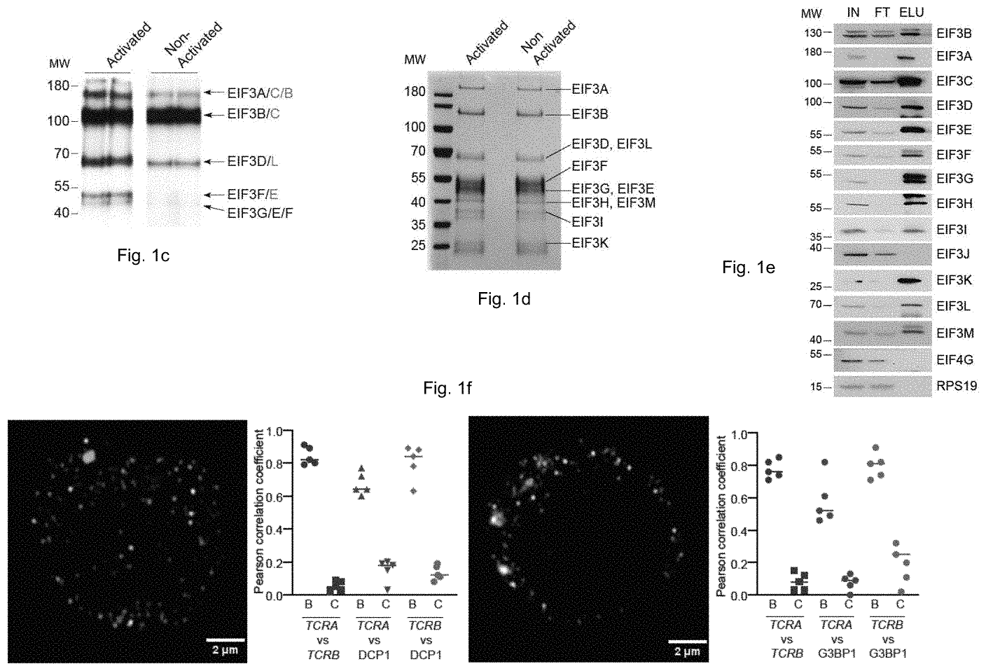

Here we describe compositions and methods for selectively increasing or decreasing the protein synthesis of engineered immune cell surface receptors using noncoding sequences in the 3′-untranslated region (3′-UTR) of messenger RNAs (mRNAs) encoding the engineered TCRs or CARs. Exogenous genes encoding engineered TCRs and CARs require nucleic acid sequences that control pre-mRNA transcription and processing steps, such as splicing and polyadenylation. Additionally, the resulting mature mRNAs must include noncoding elements at their 5′ and 3′ ends, or 5′-untranslated or 3′-untranslated (5′-UTR and 3′-UTR) sequences. See for example FIG. 1 of (Kasinath, 2006). Here we disclose 3′-UTR sequences sensitive to regulation by translation initiation factor eIF3 that can be used to modulate the strength and time duration of TCR or CAR protein synthesis.

The invention provides compositions and methods for modulating engineered immune cell receptor translation using a heterologous noncoding sequence element.

In an aspect the invention provides a method of modulating engineered immune cell receptor translation using a heterologous noncoding sequence element, comprising: providing an immune cell comprising an engineered cell surface receptor gene operably linked to a heterologous untranslated region (UTR) comprising one or more eukaryotic initiation factor 3 (eIF3) responsive sites sufficient to modulate translation of the cell surface receptor.

In embodiments:

the heterologous UTR comprises one or more deletion mutations of the one or more eIF3 responsive sites;

the heterologous UTR comprises the one or more eIF3-responsive sites embedded in a heterologous 3′ -UTR sequence;

the cell surface receptor is a chimeric antigen receptors (CAR) or a T cell receptor (TCR);

the noncoding sequences or responsive sites are in the heterologous 3′ UTR of the gene;

the noncoding sequences or responsive sites are human or derived from analogous sequences found in Hominidai, Hylobatidae, Cercopithecidae, Perissodactyla, Artiodactyla, Carnivora or Chiroptera;

the method is configured for cancer immunotherapy, known as adoptive cell therapy (ACT), or other cell-based therapies using engineered regulatory T cells (engineered Tregs) to treat immune dysfunction such as autoimmunity or organ transplant rejection;

the method further comprises the step of introducing the cell into a human host in need of a cell-based therapy; and/or

the method further comprises an antecedent step of introducing the gene into the cell, by for examples, using a CRISPR/Cas system, a MegaTal, a meganuclease or zinc-finger nucleases or TALENs.

In an aspect the invention provides a polynucleotide, comprising a heterologous untranslated region (UTR) element, said variant UTR element comprising one or more eukaryotic initiation factor 3 (eIF3) responsive sites sufficient to modulate the translation of a recombinant protein.

In embodiments:

the polynucleotide further comprises a nucleic acid encoding a recombinant protein, such as a cell surface receptor, operably linked to the heterologous eIF3-responsive UTR element;

the heterologous UTR comprises one or more deletion mutations of the one or more eIF3 responsive sites;

the heterologous UTR comprises the one or more eIF3-responsive sites embedded in a heterologous 3′-UTR sequence;

the cell surface receptor is a chimeric antigen receptors (CAR) or a T cell receptor (TCR);

the noncoding sequences or responsive sites are in the heterologous 3′ UTR of the gene; and/or

the noncoding sequences or responsive sites are human or derived from analogous sequences found in Hominidai, Hylobatidae, Cercopithecidae, Perissodactyla, Artiodactyla, Carnivora or Chiroptera.

In an aspect the invention provides a vector or cell comprising a disclosed recombinant polynucleotide.

In an aspect the invention provides an immune cell comprising an engineered receptor gene encoding an engineered receptor operably linked to a disclosed heterologous polynucleotide.

In an aspect the invention provides a cell-based composition adapted and configured for adoptive cell therapy (ACT), or other cell-based therapies using engineered regulatory T cells (engineered Tregs) to treat immune dysfunction such as autoimmunity or organ transplant rejection, and comprising an immune cell comprising an engineered receptor gene encoding an engineered receptor operably linked to a disclosed polynucleotide.

The present disclosure provides methods and compositions for translational regulation of a heterologous polypeptide (for example, a cell surface receptor) using a heterologous 3′ non-coding sequence element. The sequence element is sensitive to regulation by eIF3 translational regulation such that protein expression of fusion constructs comprising a recombinant gene encoding a receptor operably linked to the heterologous 3′ non-coding element are regulated at a translational level. The present disclosure provides recombinant nucleic acid compositions of these receptors (e.g. chimeric antigen receptor, T cell receptors and NK cell receptors), delivery means comprising the receptors, modified cells comprising the receptors, methods of making the receptors and modified cells as well as use of the modified cells for the treatment of diseases and/or disorders in subjects in need thereof.

In some aspects, the cell surface receptor may be a CAR, which may comprise (i) an antigen-specific component (“antigen binding molecule”), (ii) one or more costimulatory domains (which includes a hinge/spacer domain), and (iii) one or more activating domains. Each domain may be heterogeneous, that is, comprised of sequences derived from different protein chains. CAR-expressing immune cells (such as T cells) may be used in various therapies, including cancer and infectious disease therapies. In some aspects, CARs can be used with regulatory T cells for autoimmune disorder therapies.

In some aspects, the cell surface receptor is a T cell receptor (TCR). The nucleic acid sequences encoding a TCR of interest may be isolated from another T cell and operably linked to the heterologous 3′ UTR sequence for translational regulation. TCRs are not limited to the detection of surface antigens like antibodies, rather they recognize peptides presented on the MHC complex and have the potential to recognize the whole proteome (Walseng et al (2017) Scientific Reports 7, No. 10713). TCRs can be isolated from tumor infiltrating lymphocytes (TILs) or other types of T cells by methods known in the act (see e.g. Parkhurst et al (2014) J Immunother Cancer 2(suppl 3):P33).

Polynucleotides encoding such CARs and/or TCRs operably linked to the heterologous 3′ untranslated region may be transduced into T cells such that the receptors are expressed in T cells, e.g., a patient's own T cells or in a healthy donor T cell. When the transduced T cells are transplanted into a patient for cancer immunotherapy, the receptors can direct the T cells to recognize and bind an epitope present on the surface of cancer cells, thus, allowing binding of cancer cells rather than non-cancerous cells. This binding leads to activation of cytolytic mechanisms in the T cell that specifically kill the bound cancer cells. In some aspects, the endogenous TCRA, TCRB, TCRD and/or TCRG genes present in the T cell to be transduced are knocked out to prevent pairing between subunits of the endogenous TCR and the recombinant TCR being introduced.

Gamma delta (γδ) T cells are a lower-frequency T cell population in which the T cell receptor (TCR) is expressed from the TCRG and TCRD genes, forming TCRγδ complexes, instead of the TCRA and TCRB genes (Khairalla et al, (2018) Front Immunol 9:2636). They can mount rapid immune responses to a wide range of tissue insults, referred to as “lymphoid-stress surveillance.” Further, they can be engineered as cell immunotherapies, either by expressing TCRαβ variants or by expressing chimeric antigen receptors (CARs) (Katz and Rabinovich, (2020) Methods Mol Biol 2097:3-44). Due to their related function to the more conventional αβ T cells, the control of TCRγδ expression at the level of protein synthesis is desirable. Furthermore, the 3′-UTR elements of the TCRG and TCRD genes (encoded by TRDC and TRGC2 constant segments) can be useful to control the expression of engineered TCRs and CARs at the level of protein synthesis for use in cell therapies. The 3′-UTR elements are encoded on human chromosome 7 for TRGC2 (NG_001336.2, nucleotide range 133,925-134,368) and on human chromosome 14 for TRDC (NG_01332.3, nucleotide range 843,629-843,869). These can be added 3′ to the coding region of the mRNA encoding the engineered receptor.

Although the preferred noncoding sequences to introduce into the 3′-UTR of engineered TCRs or CARs are human sequences, one of ordinary skill in the art can obtain homologous sequences in other mammals using commonly-available sequence alignment programs such as megablast or discontiguous megablast (McGinnis, 2004). Using these sequence alignment programs, homologous sequences can be identified in mammals including Hominidae (Pongo, Pan, Gorilla, and Homo genera), Hylobatidae (lesser apes), Cercopithecidae (Old World monkeys), and more distantly-related mammals including Perissodactyla (Odd-toed ungulates), Artiodactyla (Even-toed ungulates), Carnivora including Canidae, and Chiroptera including bats. Related sequences to these 3′-UTR elements can be identified by conducting a discontiguous megablast search using the National Center for Biotechnology Information (NCBI) BLAST server with sequence NG_001336.2, nucleotide range 133925-134368, yielding homologous sequences from the above mammalian taxa with 71%-100% sequence coverage, and at least 86% sequence identity at the nucleotide level. Notably, such a search also identifies the 3′-UTR element of the human TRGC1 constant segment (also present in chromosome 7, in sequence NG_01336.2, nucleotide range 113861-116069). As another example, one can conduct a discontiguous megablast search using the National Center for Biotechnology Information (NCBI) BLAST server (and human chromosome 14 with sequence NG_01332.3, nucleotide range 843,629-843,869, yielding homologous sequences from the above mammalian taxa with 74%-100% sequence coverage, and at least 74% sequence identity at the nucleotide level.

The heterologous 3′ UTR sequence comprises sequences derived from primate TCRA or TCRB genes. In some aspects, the heterologous 3′ UTR sequence is derived from the human TCRA gene located on chromosome 14 and comprised by the following sequence:

(SEQ ID NO: 1)
5′
AAGGAGGTGAAAGCTGCTACCACCTCTGTGCCCCCCCGGCAA
TGCCACCAACTGGATCCTACCCGAATTTATGATTAAGATTGC
TGAAGAGCTGCCAAACACTGCTGCCACCCCCTCTGTTCCCTT
ATTGCTGCTTGTCACTGCCTGACATTCACGGCAGAGGCAAGG
CTGCTGCAGCCTCCCCTGGCTGTGCACATTCCCTCCTGCTCC
CCAGAGACTGCCTCCGCCATCCCACAGATGATGGATCTTCAG
TGGGTTCTCTTGGGCTCTAGGTCCTGCAGAATGTTGTGAGGG
GTTTATTTTTTTTTAATAGTGTTCATAAAGAAATACATAGTA
TTCTTCTTCTCAAGACGTGGGGGGAAATTATCTCATTA

In some aspects, the heterologous 3′ UTR sequence is derived from the human TCRB gene located on chromosome 7, and is comprised by the following sequence:

(SEQ ID NO: 2)
5′
TCACCCAGGATTCTCCTGTACCTGCTCCCAATCTGTGTTCCTA
AAAGTGATTCTCACTCTGCTTCTCATCTCCTACTTACATGAAT
ACTTCTCTCTTTTTTCTGTTTCCCTGAAGATTGAGCTCCCAAC
CCCCAA

In some aspects, the heterologous 3′ UTR sequence is derived from the human TRCD gene located on chromosome 14, and is comprised by the following sequence:

(SEQ ID NO: 3)
5′
GGCTGACTGGCATGAGGAAGCTACACTCCTGAAGAAACCAAAGG
CTTACAAAAATGCATCTCCTTGGCTTCTGACTTCTTTGTGATTC
AAGTTGACCTGTCATAGCCTTGTTAAAATGGCTGCTAGCCAAAC
CACTTTTTCTTCAAAGACAACAAACCCAGCTCATCCTCCAGCTT
GATGGGAAGACAAAAGTCCTGGGGAAGGGGGGTTTATGTCCTAA
CTGCTTTGTATGCTGTTTTATAAAGGGATAGAAGGA

In some aspects, the heterologous 3′ UTR sequence is derived from the human TRGC2 gene located on chromosome 7, and is comprised by the following sequence:

(SEQ ID NO: 4)
5′
CAGACGGTGGCACAAGGAGGCCATCTTTTCCTCATCGGTTA
TTGTCCCTAGAAGCGTCTTCTGAGGATCTAGTTGGGCTTTC
TTTCTGGGTTTGGGCCATTTCAGTTCTCATGTGTGTACTAT
TCTATCATTATTGTATAATGGTTTTCAAACCAGTGGGCACA
CAGAGAACCTCACTCTGTAATAACAATGAGGAATAGCCATG
GCGATCTCCAGCACCAATCTCTCCATGTTTTCCACAGCTCC
TCCAGCCAACCCAAATAGCGCCTGCTATAGTGTAGACAGCC
TGCGGCTTCTAGCCTTGTCCCTCTCTTAGTGTTCTTTAATC
AGATAACTGCCTGGAAGCCTTTCATTTTACACGCCCTGAAG
CAGTCTTCTTTGCTAGTTGAATTATGTGGTGTGTTTTTCCG
TAATAAGCAAAATAAATTTAAAAAAATGAAAAGT

In some aspects, the heterologous 3′ UTR sequence is derived from analogous sequences found in Hominidai, Hylobatidae, Cercopithecidae, Perissodactyla, Artiodactyla, Carnivora or Chiroptera. In some aspects, the heterologous 3′ UTR sequence comprises 1 or more mutations 15 B20-053-2US in the nucleic acid sequence while preserving its sensitivity to eIF3 translational regulation. General

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Definitions

The terms “nucleic acid,” “polynucleotide,” and “oligonucleotide” are used interchangeably and refer to a deoxynbonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, above). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2, and 100,000,000 nucleotides in length (or any integer value therebetween or thereabove), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

A “chromosome,” is a chromatin complex comprising all or a portion of the genome of a cell. The genome of a ceil is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins and fusion nucleic acids. Examples of the second type of fusion molecule include, but are not limited to, a fusion between a triplex-forming nucleic acid and a polypeptide, and a fusion between a minor groove binder and a nucleic acid.

“Gene expression” refers to the conversion of the information, contained in a gene, into a gene product, A gene product can be the direct transcriptional product of a gene (e.g., mRNA, tRNA, rRNA, anti-sense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Eukaryotic” cells include, but are not limited to, fungal cells (such as yeast), plant cells, animal cells, mammalian cells and human cells (e.g., T-cells), including stem cells (pluripotent and multipotent).

A “T effector cell” (Teff) is a CD4+ or CD8+ T cell that acts immediately to a stimulus. These cells play a central role in cellular-mediated immunity following differentiation. T cells are activated following stimulation by an antigen presenting cell and differentiate into T effector cells that perform critical effector functions such as producing cytotoxic molecules and antibodies. T effector cells migrate to the site of inflammation (e.g. infection) and produce chemokines to recruit additional immune cells.

A “regulatory T cell” (Treg) is also known as a suppressor T cell, and is a subpopulation of T cells that modulate the immune system, maintain tolerance to self-antigens and prevent autoimmune disease. Tregs are immunosuppressive and generally suppress or downregulate induction and proliferation of T effector cells. Tregs are CD25+, CD12710 and FOXP3+.

As used herein, an “immune effector cell” is a leukocyte that can modulate an immune response Immune effector cells include T cells, B cells, natural killer (NK) cells, iNKT cells (invariant T-cell receptor alpha natural killer T cells), and macrophages. T cell receptor (TCR)-bearing or CAR-bearing immune effector cells include, of course, T cells, but also cells which have been engineered to express a T cell receptor or chimeric antigen receptor Immune effector cells may be obtained or derived/generated from any appropriate source, such as including, but not limited to, healthy donors, peripheral blood mononuclear cells, cord blood, and induced pluripotent stem cells (iPSC).

As used herein, a “CAR-bearing immune effector cell” is an immune effector cell which has been transduced with at least one CAR. A “CAR-T cell” is a T cell which has been transduced with at least one CAR; CAR-T cells can be mono, dual, or tandem CAR-T cells. CAR-T cells can be autologous, meaning that they are engineered from a subject's own cells, or allogeneic, meaning that the cells are sourced from a healthy donor, and in many cases, engineered so as not to provoke a host-vs-graft or graft-vs-host reaction. Similarly, a NK-CAR cell is an NK cell which has been transduced with at least one CAR.

The term “allogeneic” refers to any material derived from a different individual of the same species as the individual to whom the material is introduced. Two or more individuals are said to be allogeneic to one another when the genes at one or more loci are not identical. In some aspects, allogeneic material from, individuals of the same species may be sufficiently unlike genetically to interact antigenically.

The term “autologous” refers to any material derived from the same individual as to whom the material is later re-introduced. In some instances, the material is modified prior to re-introduction.

The term “heterologous” refers to material (e.g. a nucleic acid, polypeptide or cell) that is not found in the native nucleic acid, polypeptide or tissue, respectively. For example, a heterologous gene may be one that is not normally found in a cell, or at a specific location in the genome. A heterologous sequence is one that is not normally found in a sequence, for example a sequence element operably linked to another wherein the elements are normally not linked.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10-6 M, less than 10-7 M, less than 10-8 M, less than 10-9 M, less than 10-10 M, less than 10-11 M, less than 10-12 M, less than 10-13 M, less than 10-14 M, or less than 10-15 M. “Affinity” refers to the strength of binding, increased binding affinity being correlated with a lower Kd.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, Phyre2, etc.), available over the world wide web. See also, e.g., Altschul et al. (1990), J. Mol. Biol. 215:403-10.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Operably linked” refers to a juxtaposition wherein the components so described are in a relationship permitting them to function in their intended manner For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, non-human primates, humans, mammalian farm animals, mammalian sport animals, and mammalian pets.

As used herein, the term “cytokine release syndrome” refers to a condition that may occur after treatment with some types of immunotherapy, such as monoclonal antibodies and CAR-T or other CAR-bearing immune effector cells. Cytokine release syndrome is caused by a large, rapid release of cytokines into the blood from immune cells affected by the immunotherapy. Symptoms of CRS include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination. CRS can manifest along a spectrum of mild to fatal, and can be ranked by severity as follows:
Grade 1 : Mild reaction, infusion interruption not indicated; intervention not indicated
Grade 2: Therapy or infusion interruption indicated but responds promptly to symptomatic treatment (e.g., antihistamines, NSAIDS, narcotics, IV fluids); prophylactic medications indicated for <=24 hrs
Grade 3: Prolonged (e.g., not rapidly responsive to symptomatic medication and/or brief interruption of infusion); recurrence of symptoms following initial improvement; hospitalization indicated for clinical sequelae (e.g., renal impairment, pulmonary infiltrates)
Grade 4: Life-threatening consequences; pressor or ventilatory support indicated
Grade 5: Death
See, e.g., “Common Terminology Criteria for Adverse Events (CTCAE) Version v4.03,” National Institutes of Health and National Cancer Institute, Jun. 14, 2010; Lee D W et al, Blood 2014 124(2): 188-95.

Nuclease and Nuclease System

In some cases, the cell surface receptor is inserted into the genome of the cell using a CRISPR/cas system or other engineered nuclease.

Examples of CRISPR/Cas effector polypeptides are CRISPR/Cas endonucleases (e.g., class 2 CRISPR/Cas effector polypeptide such as a type II, type V, or type VI CRISPR/Cas effector polypeptide). Where a CRISPR/Cas effector polypeptide has endonuclease activity, the CRISPR/Cas effector polypeptide may also be referred to as a “CRISPR/Cas endonuclease.” A CRISPR/Cas effector polypeptide can also have reduced or undetectable endonuclease activity. A CRISPR/Cas effector polypeptide can also be a fusion CRISPR/Cas effector polypeptide comprising a heterologous fusion partner. In some cases, a suitable CRISPR/Cas effector polypeptide is a class 2 CRISPR/Cas effector polypeptide. In some cases, a suitable CRISPR/Cas effector polypeptide is a class 2 type II CRISPR/Cas effector polypeptide (e.g., a Cas9 protein). In some cases, a suitable CRISPR/Cas effector polypeptide is a class 2 type V CRISPR/Cas endonuclease (e.g., a Cpf1 protein, a C2c1 protein, or a C2c3 protein). In some cases, a suitable CRISPR/Cas effector polypeptide is a class 2 type VI CRISPR/Cas effector polypeptide (e.g., a C2c2 protein; also referred to as a “Cas13a” protein). Also suitable for use is a CasX protein. Also suitable for use is a CasY protein.

In some cases, the CRISPR/Cas effector polypeptide is a Type II CRISPR/Cas effector polypeptide. In some cases, the CRISPR/Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence.

In some cases, the Cas9 polypeptide is a Campylobacter jejuni Cas9 (CjCas9) polypeptide. CjCas9 recognizes the 5′-NNNVRYM-3′ (SEQ ID NO:5) as the protospacer-adjacent motif (PAM). In some cases, a suitable Cas9 polypeptide comprises an amino acid sequence having at least 50%, at least 60%, at least 70%, at least 80%, at least 90%, at least 95%, at least 98%, at least 99%, or more than 99%, amino acid sequence identity to the CjCas9 amino acid sequence.

In some cases, a suitable Cas9 polypeptide is a high-fidelity (HF) Cas9 polypeptide. Kleinstiver et al. (2016) Nature 529:490. For example, an HF Cas9 polypeptide can comprise an amino acid sequence having at least 90%, at least 95%, at least 98%, at least 99%, or 100%, amino acid sequence identity to the amino acid sequence, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable CRISPR/Cas effector polypeptide is a type V CRISPR/Cas effector polypeptide. In some cases, a type V CRISPR/Cas effector polypeptide is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence.

In some cases, a suitable CRISPR/Cas effector polypeptide is a CasX or a CasY polypeptide. CasX and CasY polypeptides are described in Burstein et al. (2017) Nature 542:237.

Other CRISPR/Cas systems include type II CRISPR/Cas effector polypeptides (e.g., Cas9); type V-A CRISPR/Cas effector polypeptides (e.g., Cpf1 (also referred to a “Cas12a”)); type V-B CRISPR/Cas effector polypeptides (e.g., C2c1 (also referred to as “Cas12b”)); type V-C CRISPR/Cas effector polypeptides (e.g., C2c3 (also referred to as “Cas12c”)); type V-U1 CRISPR/Cas effector polypeptides (e.g., C2c4); type V-U2 CRISPR/Cas effector polypeptides (e.g., C2c8); type V-U5 CRISPR/Cas effector polypeptides (e.g., C2c5); type V-U4 CRISPR/Cas proteins (e.g., C2c9); type V-U3 CRISPR/Cas effector polypeptides (e.g., C2c10); type VI-A CRISPR/Cas effector polypeptides (e.g., C2c2 (also known as “Cas13a”)); type VI-B CRISPR/Cas effector polypeptides (e.g., Cas13b (also known as C2c4)); and type VI-C CRISPR/Cas effector polypeptides (e.g., Cas13c (also known as C2c7)).

In addition to the Cas protein, a CRISPR/Cas system may also comprise a nucleic acid comprising a nucleotide sequence encoding the CRISPR/Cas effector polypeptide guide RNA. Methods for designing appropriate guide RNAs are known in the art.

In some cases, the nuclease is a “chimeric nuclease”. In some cases, a chimeric zinc finger protein (ZFP) or a chimeric transcription activator like effector protein (TALE) or a megaTAL is delivered to the cell using methods known in the art. In some cases, the ZFP protein comprises a nuclease domain (e.g. a FokI nuclease domain, for example a zinc finger nuclease ZFN) and is delivered to a cell or organism comprising a cell such that the gene recognized by the ZFP DNA binding domain is cleaved. In some cases, the TALE protein or megaTAL protein comprises a nuclease domain (e.g. a FokI nuclease domain, for example a TALEN or MegaTAL) and is delivered to a cell or organism comprising a cell such that the gene recognized by the TALE DNA binding domain is cleaved.

### Cell Surface Receptor Gene

In some cases, a system of the present disclosure comprises a donor nucleic acid. By a “donor nucleic acid” or “donor sequence” or “donor polynucleotide” or “donor template” it is meant a nucleic acid sequence to be inserted at the site cleaved by a CRISPR/Cas effector protein or engineered nuclease (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

In some cases, the present disclosure provides a modified cell surface receptor wherein the receptor is a CAR, a TCR or an ACTR. In some cases, the cell surface receptor further comprises a heterologous 3′ UTR sequence. In some cases, the heterologous 3′ UTR sequence is comprised by the following sequence:

(SEQ ID NO: 6)
5′
AAGGAGGTGAAAGCTGCTACCACCTCTGTGCCCCCCCGGCAA
TGCCACCAACTGGATCCTACCCGAATTTATGATTAAGATTGC
TGAAGAGCTGCCAAACACTGCTGCCACCCCCTCTGTTCCCTT
ATTGCTGCTTGTCACTGCCTGACATTCACGGCAGAGGCAAGG
CTGCTGCAGCCTCCCCTGGCTGTGCACATTCCCTCCTGCTCC
CCAGAGACTGCCTCCGCCATCCCACAGATGATGGATCTTCAG
TGGGTTCTCTTGGGCTCTAGGTCCTGCAGAATGTTGTGAGGG
GTTTATTTTTTTTTAATAGTGTTCATAAAGAAATACATAGTA
TTCTTCTTCTCAAGACGTGGGGGGAAATTATCTCATTA

In some aspects, the heterologous 3′ UTR sequence is comprised by the following sequence:

(SEQ ID NO: 7)
5′
TCACCCAGGATTCTCCTGTACCTGCTCCCAATCTGTGTTCCTA
AAAGTGATTCTCACTCTGCTTCTCATCTCCTACTTACATGAAT
ACTTCTCTCTTTTTTCTGTTTCCCTGAAGATTGAGCTCCCAAC
CCCCAA

In some aspects, the heterologous 3′ UTR sequence is comprised by the following sequence:

(SEQ ID NO: 8)
5′
GGCTGACTGGCATGAGGAAGCTACACTCCTGAAGAAACCA
AAGGCTTACAAAAATGCATCTCCTTGGCTTCTGACTTCTTTGT
GATTCAAGTTGACCTGTCATAGCCTTGTTAAAATGGCTGCTAG
CCAAACCACTTTTTCTTCAAAGACAACAAACCCAGCTCATCCT
CCAGCTTGATGGGAAGACAAAAGTCCTGGGGAAGGGGGGTTTA
TGTCCTAACTGCTTTGTATGCTGTTTTATAAAGGGATAGAAGGA

In some aspects, the heterologous 3′ UTR sequence is comprised by the following sequence:

(SEQ ID NO: 9)
5′
CAGACGGTGGCACAAGGAGGCCATCTTTTCCTCATCGGTTATT
GTCCCTAGAAGCGTCTTCTGAGGATCTAGTTGGGCTTTCTTTC
TGGGTTTGGGCCATTTCAGTTCTCATGTGTGTACTATTCTATC
ATTATTGTATAATGGTTTTCAAACCAGTGGGCACACAGAGAAC
CTCACTCTGTAATAACAATGAGGAATAGCCATGGCGATCTCCA
GCACCAATCTCTCCATGTTTTCCACAGCTCCTCCAGCCAACCC
AAATAGCGCCTGCTATAGTGTAGACAGCCTGCGGCTTCTAGC
CTTGTCCCTCTCTTAGTGTTCTTTAATCAGATAACTGCCTGGA
AGCCTTTCATTTTACACGCCCTGAAGCAGTCTTCTTTGCTAGT
TGAATTATGTGGTGTGTTTTTCCGTAATAAGCAAAATAAATTT
AAAAAAATGAAAAGT

### Genome-Edited T Cells and CAR-T Cells

A CAR-T cell is a T cell which expresses a chimeric antigen receptor. The T cell expressing a CAR molecule may he a helper T cell, a cytotoxic T cell, a viral-specific cytotoxic T cell, a memory T cell, or a gamma delta (gd) T cell. In some aspects of the present invention, provided is a chimeric antigen receptor comprising a heterologous 3′ UTR as disclosed herein.

A chimeric antigen receptor (CAR), is a recombinant fusion protein comprising: 1) an extracellular ligand-binding domain, i.e., an antigen-recognition domain, 2) a transmembrane domain, and 3) a signaling transducing domain.

The extracellular ligand-binding domain is an oligo- or polypeptide that is capable of binding a ligand. Preferably, the extracellular ligand-binding domain will be capable of interacting with a cell surface molecule which may be an antigen, a receptor, a peptide ligand, a protein ligand of the target, or a polypeptide of the target. The extracellular ligand-binding domain can specifically bind to an antigen with an affinity constant or affinity of interaction (KD) between about 0.1 pM to about 10 pM, to about 0.1 pM to about 1 pM, or to about 0.1 pM to about 100 nM. Methods for determining the affinity constant or affinity of interaction (KD) are well-known in the art. In some instances, the extracellular ligand-binding domain is chosen to recognize a ligand that acts as a cell surface marker on target cells associated with particular disease states.

The signal peptide of the present disclosure directs the appended polypeptide, i.e., the CAR receptor, to the cell membrane wherein the extracellular ligand-binding domain of the appended polypeptide is displayed on the cell surface, the transmembrane domain of the appended polypeptide spans the cell membrane, and the signaling transducing domain of the appended polypeptide is m the cytoplasmic portion of the cell in one embodiment, the signal peptide is the signal peptide from human CD8a. In one embodiment, the signal peptide is a functional fragment of the CD8a signal peptide. A functional fragment is defined as a fragment of at least 10 amino acids of the CD8a signal peptide that directs the appended polypeptide to the cell membrane and/or cell surface.

Typically, the extracellular ligand-binding domain is linked to the signaling transducing domain of the chimeric antigen receptor (CAR) by a transmembrane domain (Tm). The transmembrane domain traverses the cell membrane, anchors the CAR to the T cell surface, and connects the extracellular ligand-binding domain to the signaling transducing domain, impacting the expression of the CAR on the T cell surface.

The distinguishing feature of the transmembrane domain in the present disclosure is the ability to be expressed at the surface of an immune cell to direct an immune cell response against a pre-defined target cell. The transmembrane domain can be derived from natural or synthetic sources. Alternatively, the transmembrane domain of the present disclosure may be derived from any membrane-bound or transmembrane protein.

Non-limiting examples of transmembrane polypeptides of the present disclosure include the subunits of the T-cell receptor such as a, b, g, or z, polypeptides, constituting the CD3 complex, IL-2 receptor p55 (a chain), p75 (b chain or y chain), and subunit chains of the Fc receptors, in particular the FcyIII or CD proteins. Alternatively, the transmembrane domain can be synthetic and comprise predominantly hydrophobic amino acid residues (e.g., leucine and valine). In one embodiment, the transmembrane domain is derived from the T-cell surface glycoprotein CD8 alpha chain isoform 1 precursor (NP_001 139345.1) selected from CD8a, and CD28.

The transmembrane domain can further comprise a hinge region between extracellular ligand-binding domain and said transmembrane domain. The term “hinge region” generally means any oligo- or polypeptide that functions to link the transmembrane domain to the extracellular ligand-binding domain. In particular, hinge region is used to provide more flexibility and accessibility for the extracellular ligand binding domain. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), QX-40 (CD 134), EB3z, the T cell receptor a orb chain, CD45, CD4, CDS, CDS, CD8a, CD9, CD16, CD22, CD33, CD37, CD64, CD80, CD86, iCQS, CD154 or from all or parts of an antibody constant region.

Alternatively, the hinge region may be a synthetic sequence that corresponds to a naturally-occurring hinge sequence or the hinge region may be an entirely synthetic hinge sequence. In one embodiment, the hinge domain comprises a part of human CD8a, FcyRIIIa receptor, or IgG1, and referred to in this specification as, and have at least 80%, 90%, 95%, 97%, or 99% sequence identity with these polypeptides.

Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM that can be used in the present disclosure can include those derived from TCR-z, FcRy, FcR-b, FcRs, CD3y, CD35, CD3s, CDS, CD22, CD79a, CD79b and CD66d. In some embodiments, the signaling transducing domain of the CAR can comprise the CD3z signaling domain with an amino acid sequence of at least 80%, 90%, 95%, 97%, or 99% sequence identity thereto.

A chimeric antigen receptor (CAR) of the present disclosure comprises a signal transducing domain or intracellular signaling domain of a CAR which is responsible for intracellular signaling following the binding of the extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper T cell activity, including the secretion of cytokines. Thus, the term “signal transducing domain” refers to the portion of a protein which transduces the effector signal function signal and directs the cell to perform a specialized function.

In one embodiment, the extracellular ligand-binding domain comprises a single chain antibody fragment (scFv) comprising the light (VL) and the heavy (VH) variable fragment joined by a linker (e.g., GGGGS) and confers specificity for either a T cell antigen or an antigen that is not specific to a T cell. In one embodiment, the chimeric antigen receptor of a CAR-T cell may bind to an T cell-specific antigen expressed or overexpressed on a malignant T cell for which a CAR-T cell is deficient in the antigen (e.g., a genome-edited CAR-T cell).

In some embodiments, the provided binding molecule is a T cell receptor (TCR) or antigen-binding fragment thereof. In some aspects of the present invention, provided is a TCR comprising a heterologous 3′ UTR as disclosed herein. In some embodiments, a “T cell receptor” or “TCR” is a molecule that contains variable a and b chains (also known as TCRα and TCRβ, respectively) or variable g and d chains (also known as TCRγ and TCRδ, respectively), or antigen-binding portions thereof, and which is capable of specifically binding to an antigen, e.g., a peptide antigen or peptide epitope bound to an MHC molecule. In some embodiments, the TCR is in the ab form. In some embodiments the TCR is in the gd form. Typically, TCRs that exist in ab and gd forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens, such as peptides bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, such as a TCR containing the a chain and b chain. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a (Va) chain and variable b (Vp) chain of a TCR, or antigen-binding fragments thereof sufficient to form a binding site for binding to a specific MHC-peptide complex.

In some embodiments, the variable domains of the TCR contain complementarity determining regions (CDRs), which generally are the primary contributors to antigen recognition and binding capabilities and specificity of the peptide, MHC and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores el al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the b-chain can contain a further hypervariable region (CDR4 or HVR4), which generally is involved in superantigen binding and not antigen recognition (Kotb (1995) Clinical Microbiology Reviews, 8:411-426).

In some embodiments, the a-chain and/or b-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex.

In some aspects, provided herein are cells comprising an immune receptor (e.g. a CAR and/or TCR) wherein then gene encoding said immune receptor comprises a heterologous 3′ UTR as disclosed herein. In some cases, the cell is an immune effector cell. In some cases, the immune receptor comprises a heterologous 3′ UTR derived from a TCRA, TCRB, TCRC or TRCDG2 gene.

As used herein, an ACTR polypeptide or construct refers to a non-naturally occurring molecule that can be expressed on the surface of a host cell and comprises an extracellular domain (e.g., a CD16A extracellular domain) capable of binding to a target molecule containing an Fc portion and one or more cytoplasmic signaling domains for triggering effector functions of the immune cell expressing the ACTR polypeptide, wherein at least two domains of the ACTR polypeptide may be derived from different molecules. The ACTR polypeptide may comprise a CD16A extracellular domain capable of binding to a target molecule containing an Fc portion, a transmembrane domain, one or more co-stimulatory signaling domains, and a CD3 cytoplasmic signaling domain. At least one of the co-stimulatory signaling domains may be a CD28 co-stimulatory domain. The ACTR polypeptide can either be free of a hinge domain from any non-CD 16A receptor or comprise more than one co-stimulatory signaling domain if the transmembrane domain is a CD8 transmembrane domain.

Antibodies for use with the described ACTR polypeptide can bind to a protein on the surface of a target cell (e.g., a cancer cell) Immune cells that express receptors capable of binding such Fc-containing molecules, for example the ACTR polypeptide molecules described herein, recognize the target cell-bound antibodies and this receptor/antibody engagement stimulates the immune cell to perform effector functions such as release of cytotoxic granules or expression of cell-death-inducing molecules, leading to enhanced cell toxicity of the target cells.

NK cells are innate immune effectors with the ability to exert rapid cytotoxicity against cancer and virus-infected cells without prior sensitization. NK cell functions, including degranulation, cytokine release, and cytotoxicity, are governed by a balance between signals received from inhibitory receptors (for example, the killer Ig-like receptors [KIRs] and the heterodimeric C-type lectin receptor [NKG2A]) and activating receptors (in particular, the natural cytotoxicity receptors [NCRs] NKp46, NKp30, NKp44, and the C-type lectin-like activating immunoreceptor NKG2D7) that recognize ligands on their cellular targets. These receptors therefore require mechanisms to prevent unintentional activation against normal tissues, referred to as “tolerance to self.” NK cells can also be engineered to express a CAR. NK cells are not able to mount a graft versus host response and so may have advantages for used in cell based therapies as compared with CAR-T cells (Rezvani et al (2017) Mol Ther 25(8):1769-1781).

In some aspects, the immune effector cell is a T cell. In some aspects, the immune effector cell is an NK cell. In some cases, the cell is a stem cell (e.g. a hematopoietic stem cell). In some cases, the cell is isolated from a subject, modified with a gene encoding an immune receptor comprising the heterologous 3′ UTR, and then returned to the subject (autologous cell). In some cases, the cell is isolated from a healthy volunteer, modified with a gene encoding an immune receptor comprising the heterologous 3′ UTR and then used to treat a patient in need thereof (allogenic cell). In some cases, the cell comprising the immune receptor comprising the heterologous 3′ UTR further comprises additional modifications. In some cases, the additional modifications can comprise modifications of self-recognition antigens (for example MHC antigens) and/or modifications of checkpoint inhibitor genes (for example PD1, PD1-L and/or CTLA4).

### Delivery

The nucleases and/or polynucleotides (e.g., cell surface receptors) and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, etc.).

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 8,586,526; 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more protein-encoding sequences. Thus, when one or more cell surface receptors are introduced into the cell, sequences encoding the CRISPR/Cas system or other engineered nucleases may be carried on the same vector or on different vectors. When multiple vectors are used, each vector may comprise a sequence encoding one or multiple genes.

Conventional viral and non-viral based gene transfer methods can be used to introduce nucleic acids encoding the engineered cell surface receptors in cells (e.g., mammalian cells) and target tissues. Such methods can also be used to administer nucleic acids encoding such cell surface receptors to cells in vitro. In certain embodiments, nucleic acids encoding the cell surface receptors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. Vectors suitable for introduction of transgenes into immune cells (for example T-cells) include non-integrating lentivirus vectors. See, for example, Ory et al. (1996) Proc. Natl. Acad. Sci. USA 93: 11382-11388; Dull et al. (1998) J. Virol. 72:8463-8471; Zuffery et al. (1998) J. Virol. 72:9873-9880; Follenzi et al. (2000) Nature Genetics 25:21'-222.

Methods of non-viral delivery of nucleic acids include electroporation, lipofection, microinjection, biolistics, virosomes, liposomes, immunoliposomes, polycation or lipid:nucleic acid conjugates, ribonucleoproteins (RNPs), naked DNA, naked RNA, artificial virions, and agent-enhanced uptake of DNA. Sonoporation using, e.g., the Sonitron 2000 system (Rich-Mar) can also be used for delivery of nucleic acids. In some aspects, one or more nucleic acids are delivered as mRNA. In some cases, capped mRNAs are used to increase translational efficiency and/or mRNA stability. Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™ and Lipofectamine™ RNAiMAX). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

### Description of Particular Embodiments of the Invention

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Examples of Non-limiting Aspects of the Disclosure

Aspects, including embodiments, of the present disclosure described above may be beneficial alone or in combination, with one or more other aspects or embodiments. Without limiting the foregoing description, certain non-limiting aspects of the disclosure numbered 1-36 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A nucleic acid comprising a nucleotide sequence encoding a polypeptide wherein said nucleic acid further comprises one or more heterologous untranslated elements.

Aspect 2. The nucleic acid of Aspect 1 wherein the nucleic acid is DNA or RNA.

Aspect 3. The nucleic acid of Aspect 1 wherein the polypeptide encodes a receptor.

Aspect 4. The nucleic acid of Aspect 3 wherein the encoded receptor is a T cell receptor (TCR), chimeric antigen receptor (CAR) or natural killer cell receptor (NKR).

Aspect 5. The nucleic acid of Aspect 1 wherein the one or more heterologous untranslated element is/are located in an untranslated region of the nucleic acid.

Aspect 6. The nucleic acid of Aspect 5 wherein a heterologous untranslated element comprises one or more eIF3 responsive elements.

Aspect 7. The nucleic acid of Aspect 6 wherein the one or more eIF3 responsive elements are located in the 3′ untranslated region of the nucleic acid.

Aspect 8. The encoded receptor of Aspect 4 wherein the TCR, CAR or NKR is engineered.

Aspect 9. The eIF3 responsive element of Aspect 6 wherein the element is mammalian.

Aspect 10. The eIF3 responsive element of Aspect 9 wherein the element is Hominidai, Hylobatidae, Cercopithecidae, Perissodactyla, Artiodactyla, Carnivora or Chiroptera in origin.

Aspect 11. A nucleic acid of Aspect 6 wherein the eIF3 responsive element that has 70% sequence identity or greater to the element of Aspect 10.

Aspect 12. A vector comprising the nucleic acid of Aspect 1.

Aspect 13. The vector of Aspect 12 wherein the vector is selected from a plasmid, a particle or a viral vector.

Aspect 14. A cell comprising the nucleic acid of Aspect 1.

Aspect 15. The cell of Aspect 14 wherein the nucleic acid is inserted into the genome of the cell or is not inserted into the genome of the cell.

Aspect 16. The cell of Aspect 15 wherein the nucleic acid is inserted into the genome of the cell using a CRISPR/Cas system, zinc finger nucleases, TALENs, MegaTals or a meganuclease.

Aspect 17. The cell of Aspect 14 wherein the cell is a mammalian cell.

Aspect 18. The cell of Aspect 14 wherein the cell is an autologous cell or an allogenic cell.

Aspect 19. The cell of Aspect 17 or 18 wherein the cell is selected from a T cell, a B cell, an NK cell or a stem cell.

Aspect 20. The T cell of Aspect 19 wherein the T cell is selected from an effector T cell or a regulatory T cell.

Aspect 21. The T cell of Aspect 19 wherein the T cell comprises further genomic modifications including insertions and/or deletions.

Aspect 22. The T cell of Aspect 21 wherein the insertions and/or deletions result in the knock out of one or more endogenous genes.

Aspect 23. The T cell of Aspect 22 wherein the knocked out genes encode polypeptides selected from checkpoint inhibitors, cytokine receptors, endogenous TCRs, and histocompatibility antigens.

Aspect 24. The T cell of Aspect 21 wherein insertions include insertion of a heterologous donor sequence encoding a polypeptide.

Aspect 25. The stem cell of Aspect 19 wherein the stem cell is a hematopoietic stem cell.

Aspect 26. The stem cell of Aspect 19 wherein the stem cell comprises further genomic modifications including insertions and/or deletions.

Aspect 27. A composition comprising the cell of Aspect 14.

Aspect 28. A method for modulating the translation of a polypeptide in a cell using a heterologous non-coding sequence element comprising: providing a cell comprising a gene encoding a polypeptide wherein said gene comprises one or more heterologous untranslated regions (UTRs).

Aspect 29. The method of Aspect 28 wherein the polypeptide is a receptor.

Aspect 30. The method of Aspect 29 wherein the receptor is selected from a T cell receptor (TCR), chimeric antigen receptor (CAR) or natural killer cell receptor (NKR).

Aspect 31. The method of Aspect 28 wherein the one or more heterologous untranslated element is/are located in an untranslated region (UTR) of the nucleic acid.

Aspect 32. The method of Aspect 28 wherein said UTR comprises one or more eIF3 responsive elements.

Aspect 33. The method of Aspect 32 wherein the one or more eIF3 responsive elements are located in the 3′ UTR of the nucleic acid.

Aspect 34. The method of Aspect 28 wherein the cell is selected from a T cell, a B cell, an NK cell or a stem cell.

Aspect 35. The use of the method of Aspect 28 and/or the composition of Aspect 27 in cancer immunotherapy for adoptive cell therapy (ACT), or other cell-based therapies to treat immune dysfunction or organ transplant rejection.

Aspect 36. The method of Aspect 35 further comprising the step of introducing the cell into a human host in need thereof.

The invention encompasses all combination of the particular embodiments recited herein, as if each combination had been laboriously recited.

### EXAMPLES

### Modulation of Engineered Immune Cell Receptor Translation Using Noncoding Sequence Elements

To identify candidate mRNAs that directly interact with eIF3 during early events of T cell activation, we used Jurkat cells stimulated for 5 hours with ionomycin and phorbol 12-myristate 13-acetate (I+PMA) (FIG. 1a). Jurkat cells were used as a model T cell as PAR-CLIP experiments require a large amount of cells supplemented with 4-thiouridine at a non toxic concentration (Huppertz, 2014) and Jurkat cells have a defined clonal endogenous T cell receptor and transcriptome, such that the donor to donor variability exhibited in primary T cells can be avoided. To identify eIF3 binding sites on target RNA on a transcriptome-scale we used the PAR-CLIP (Photoactivatable-Ribonucleoside-Enhanced Crosslinking and Immunoprecipitation) method. With PAR-CLIP one can identify RNA that directly interacts with an RNA-binding protein of interest. Prior to crosslinking to the RNA-binding protein (RBP), all the cellular RNA is labelled with low levels of 4-thiouridine (4SU), such that when cells are irradiated with UVA or UVB light (>310 nm) the labeled RNA will create photoadducts with the interacting RBP. The advantages of using PAR-CLIP are a higher crosslinking efficiency compared to 254 nm CLIP, and diagnostic T-to-C transitions in the sequence at positions where the RBP crosslinks to the RNA. The characteristic T-to-C mutations are introduced during the reverse transcription step in the DNA sequencing library preparation. This helps to identify the exact RNA-RBP interaction site with nucleotide resolution (Danan, 2016). The PAR-CLIP experiment of eIF3 in Jurkat cells was carried out as described in (Danan, 2016; Lee, 2015) with modifications for Jurkat cells, in two biological replicates (FIG. 1a). A PAR-CLIP of eIF3 in non activated Jurkat cells was also performed as a control experiment. In Jurkat cells, eIF3 interacted with a completely new suite of mRNAs not observed to interact with eIF3 in HEK293T cells, revealing the cell type specificity of eIF3 translation regulation. In the Jurkat PAR-CLIP experiments, RNA crosslinked to eight of the thirteen eIF3 subunits, as identified by mass spectrometry: subunits EIF3A, EIF3B, EIF3D and EIF3G as seen in HEK293T cells (Lee 2015, ibid) as well as subunits EIF3C, EIF3E, EIF3F, and EIF3L. Subunits EIF3A and EIF3B had substantially more bound mRNA than EIF3D, EIF3F and EIF3E in activated Jurkat cells. Furthermore, eIF3 in the control cells (non activated Jurkat cells) was bound to significantly fewer mRNAs than in activated Jurkat cells indicating that eIF3 is involved in specifically regulating translation of mRNAs important for T cell activation and function. These mRNAs were enriched for those encoding proteins central to immune cell function (see Table 1 listing mRNAs with highest reads detected below). Moreover, eIF3 crosslinking did not correlate with mRNA abundance.

TABLE 1
eIF3 Crosslinking mRNAs in Jurkat cells

| Gene | Total | Gene | Total | Gene | Total |
|---|---|---|---|---|---|
| Name | Reads | Name | Reads | Name | Reads |
| DUSP2 | 4300 | PFN1 | 700 | CD82 | 514 |
| SOX4 | 3632 | WNK1 | 696 | SAPCD2 | 513 |
| KMT2D | 2859 | BACH2 | 685 | TUBB | 513 |
| SLC7A5 | 2605 | C16orf72 | 685 | HIST1H3G | 507 |
| UBE2S | 2600 | PPP6R1 | 683 | HDGF | 505 |
| EGR1 | 2146 | UBN1 | 682 | BRD2 | 503 |
| LEF1 | 2115 | ADAR | 660 | PATZ1 | 503 |
| CHST2 | 2042 | ZC3HAV1 | 656 | CDKN1B | 500 |
| PIM3 | 1880 | REXO1 | 650 | CTDNEP1 | 500 |
| MIDN | 1824 | H2AFX | 648 | MFHAS1 | 498 |
| H1FX | 1703 | ZNF316 | 642 | CTBP1 | 497 |
| CDK6 | 1645 | C6orf62 | 637 | MAP2K3 | 496 |
| TNRC18 | 1627 | VCP | 636 | RMND5A | 494 |
| TRIM28 | 1600 | HCFC1 | 618 | RDH10 | 490 |
| HNRNPA0 | 1367 | PLEC | 614 | CRKL | 488 |
| JUND | 1349 | FBXL19 | 603 | ING1 | 488 |
| MAZ | 1307 | CREBBP | 602 | UBE2R2 | 481 |
| VGF | 1191 | TLE3 | 597 | HIST2H3C | 476 |
| MBD3 | 1177 | IRF1 | 594 | CD82 | 514 |
| HNRNPAB | 1163 | STK11 | 594 | SAPCD2 | 513 |
| NR4A1 | 1159 | EIF5A | 592 | TUBB | 513 |
| LAT | 1057 | HIST1H4H | 578 | HIST1H3G | 507 |
| U2AF2 | 1051 | SF1 | 577 | HDGF | 505 |
| ARF6 | 1026 | RUNX1 | 570 | BRD2 | 503 |
| PTMA | 992 | FYN | 569 | PATZ1 | 503 |
| ARID1A | 959 | TBC1D10B | 563 | CDKN1B | 500 |
| ANKRD11 | 947 | PKM | 558 | CTDNEP1 | 500 |
| EIF1 | 946 | SET | 558 | MFHAS1 | 498 |
| HIST2H3A | 938 | MACF1 | 557 | CTBP1 | 497 |
| CHSY1 | 880 | ZFAND5 | 548 | MAP2K3 | 496 |
| RBM15B | 815 | PRRC2A | 544 | RMND5A | 494 |
| MKI67 | 794 | UBE2M | 543 | RDH10 | 490 |
| CSK | 762 | ATP2A2 | 542 | CRKL | 488 |
| ZFP36L1 | 751 | ACTB | 535 | ING1 | 488 |
| EP300 | 749 | CBX6 | 530 | UBE2R2 | 481 |
| CENPB | 748 | CDKN2C | 523 | HIST2H3C | 476 |
| RBM39 | 745 | UBALD2 | 523 | PKD1 | 474 |
| IER2 | 741 | MTA2 | 518 | CAPN15 | 472 |
| HIST2H2AA4 | 733 | TMPO | 516 | KLF13 | 467 |

The identified genes were also searched for those involved in immune system processes, with the results shown below in Table 2.

TABLE 2
eIF3 cross linked mRNAs associated with immune system processes

| | | | | | | |
|---|---|---|---|---|---|---|
| ACIN1 | ACTB | ACTG1 | AGPAT5 | ARPC5 | ATP6V0C | B2M |
| BAP1 | BCL2 | BCR | CALR | CANX | CBFB | CBL |
| CCND3 | CD28 | CD3D | CNPY3 | CREBBP | CRKL | CSK |
| DCTN5 | DIAPH1 | EGR1 | EP300 | ETS1 | EVL | FYN |
| GAPDH | GNL1 | GRB2 | HIST2H2BE | HLA-C | HLA-E | HMGB1 |
| HMGB2 | HSP90AB1 | ILF3 | IRAK1 | IRF1 | ITM2A | ITPKB |
| KLC1 | LAMTOR1 | LAT | LCK | LEF1 | LMO4 | LTB |
| MAP2K1 | MAPK1 | MAPKAPK2 | MIF | MKNK2 | MLEC | MSN |
| NFKB1 | ORAI1 | PATZ1 | PIP4K2A | PKM | PMAIP1 | POLR3H |
| PPP1R14B | PRDX1 | PRKACA | PRKD2 | PSMD14 | PSMD3 | RAB10 |
| RAB14 | RAB43 | RAB7A | RAC1 | RAP2B | REL | RGCC |
| RHOA | RHOF | SATB1 | SETD2 | SLC7A5 | SOX4 | SP3 |
| SPG21 | STAT6 | STK11 | STOML2 | SURF4 | SYNCRIP | TCF3 |
| TCF7 | TNFAIP3 | TRIM27 | TRIM28 | TRIM8 | TSPAN14 | TUBB |
| VAPA | VCP | VIM | WDR1 | YTHDF2 | ZC3HAV1 | ZFP36L1 |

In HEK293T cells, eIF3 was found by PAR-CLIP to interact mainly with single discrete sites in the 5′-UTR of select mRNAs (Lee, 2015). By contrast, in activated Jurkat cells we see a multitude of cross linking patterns across eIF3-bound mRNAs indicating varied functions of eIF3 important for T cell activation and function. We focused on mRNAs that have eIF3 cross-linking sites across the entire length of the mRNA from 5′-UTR to 3′-UTR (FIG. 1b) as this pattern of cross-linking to eIF3 has not been observed before. Remarkably, the TCRA and TCRB mRNAs, which encode the alpha and beta subunits of the TCR, have this crosslinking pattern (FIG. 1b).

Crosslinking in PAR-CLIP experiments requires direct interaction between the RNA and protein of interest (Ascano et al (2012) Wiley Interdiscip Rev RNA 3:159-177). Thus, the pan-mRNA pattern of crosslinking between eIF3 and certain mRNAs indicates formation of ribonucleoprotein complexes (RNPs) highly enriched in eIF3. Remarkably, the pan-mRNA crosslinking pattern in the TCRA and TCRB mRNAs occurs in activated but not non-activated Jurkat cells. As previously known RNPs such as processing bodies (P bodies) and stress granules contain translationally repressed mRNAs, we performed fluorescence in situ hybridization (FISH) on both TCRA and TCRB mRNAs in activated Jurkat cells. Since Jurkat cells have a defined TCR, we were able to design FISH probes across the entire TCRA and TCRB transcripts. We also probed for P body and stress granule proteins DCP1 and G3BP1, respectively (Hubstenberger et al (2017) Mol Cell 68:144-157.e5; Aizer et al (2014) J Cell Sci 127: 4443-4456; Aulas et al (2015) J Cell Biol 209:73-84; Protter & Parker (2016) Trends Cell Biol 26:668-679). Both the TCRA and TCRB mRNAs formed distinct puncta, but did not co-localize with either P bodies or stress granules, or with each other (FIG. 1f). Taken together, these results indicate that TCRA and TCRB mRNA puncta represent eIF3-mRNA concentrated regions that act as translation “hot spots.”

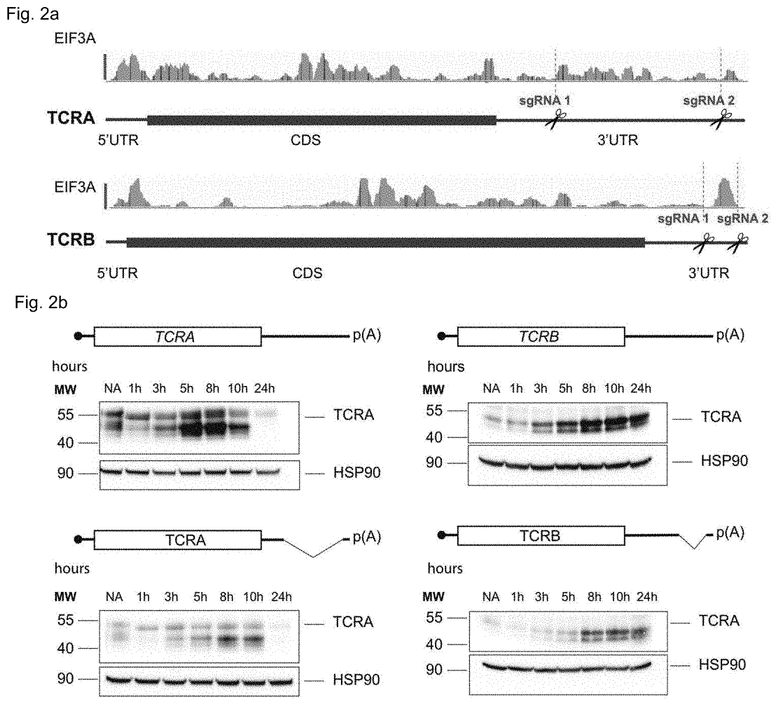

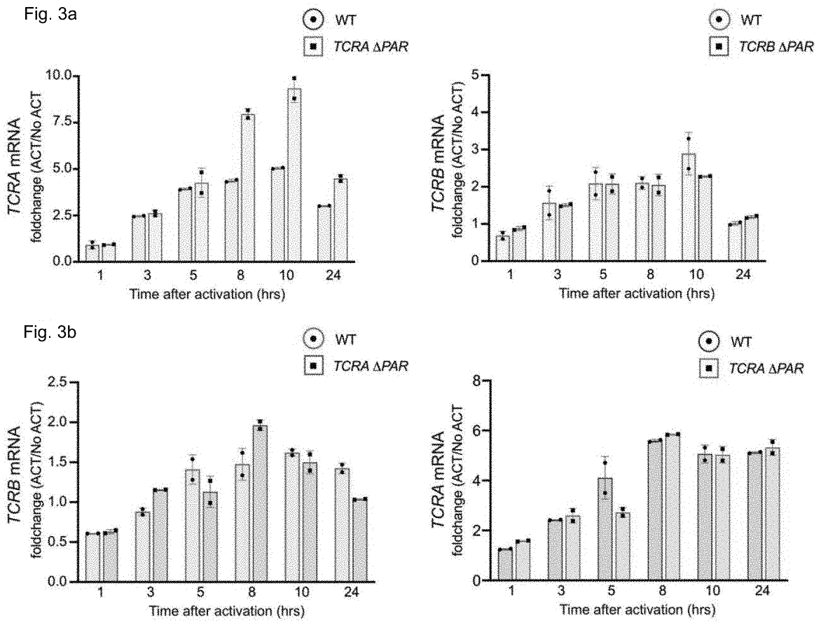

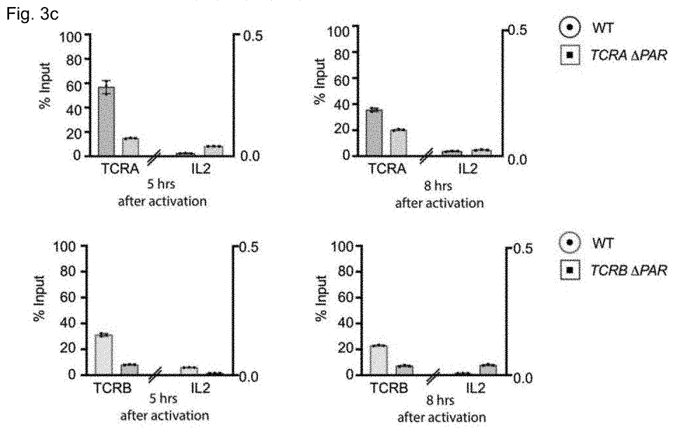

Analysis of the eIF3 crosslinking to TCRA and TCRB mRNAs identified eIF3 binding sites in the 3′-UTRs (FIG. 1b). To test these sites of eIF3 interaction, we first designed single guide RNAs (sgRNAs) that target the two boundaries of the eIF3 PAR-CLIP sites in the 3′-UTR (FIG. 2a). We then complexed both these sgRNAs with Cas9 to form sgRNA/Cas9 ribonucleoprotein particles (RNPs) and electroporated these RNPs into Jurkat cells so that the entire PAR-CLIP site would be deleted. The cells with deletion of the eIF3 PAR-CLIP site in the TCRA 3′-UTR or in the TCRB 3′-UTR (TCRA ΔPAR or TCRB ΔPAR, respectively) were confirmed by PCR analysis and then a clonal population was obtained by single cell sorting. We first measured the total protein levels of both TCRA and TCRB by western blot analysis at different time points after activation by anti-CD3 and anti-CD28 antibodies (FIG. 2b). We observed that in the TCRA ΔPAR cells, TCRA protein levels are clearly reduced, especially at the early time points after stimulation. We also observed a very clear temporal regulation of TCRA protein levels upon activation in the WT cells while in the TCRA ΔPAR cells, this regulation was not visible even when the protein levels increased at the later time points. We also measured the change in mRNA levels (activated vs non activated cells) at these time points (FIG. 3a), in both WT and the TCRA ΔPAR cell lines, to test if the failure to achieve efficient translation is caused by defects in transcription. The qPCR results showed that TCRA and TCRB mRNA levels were unaffected or even increased in the TCRA ΔPAR cells, relative to the WT cells, indicating that the 3′-UTR of the TCRA mRNA contributes to eIF3 mediated translation regulation.

To measure TCRB protein levels we used an anti-TCRA antibody as a reference as we failed to find a good working antibody against TCRB, and formation of an intact TCR is required to stabilize both subunits (Ohashi, 1985; Koning, 1988). As observed in FIG. 2b TCRA levels in the TCRB ΔPAR cells were reduced, as observed in the TCRA ΔPAR cells. Furthermore, TCRA and TCRB mRNA levels were unaffected in the TCRB ΔPAR cells (FIG. 3a). These results indicates that the PAR-CLIP site in the 3′-UTR of TCRB mRNA is also important for translation regulation, similar to TCRA mRNA.

To assess whether TCRA and TCRB translation regulation mediated by the PAR-CLIP sites in their respective 3′-UTR elements involves eIF3 binding, we measured the amount of mRNA bound to eIF3 in both WT and the TCRA ΔPAR and TCRB ΔPAR cells, 5 hours and 8 hours after activation with anti-CD3/anti-CD28 antibodies (FIG. 3b). The amount of TCRA and TCRB mRNA interacting with eIF3 was higher in WT cells than in the TCRA ΔPAR and TCRB ΔPAR cells at both 5 hours and 8 hours indicating that deleting the PAR-CLIP site disrupts eIF3 interaction with these mRNAs. Interestingly the levels of eIF3-mRNA interaction closely mirrored the total TCRA protein levels at both 5 hours and 8 hours for both WT and TCRA ΔPAR and TCRB ΔPAR cells, indicating that eIF3 interactions with the 3′-UTR sequences in the TCRA and TCRB mRNAs is tightly coupled with TCRA and TCRB protein translation levels.

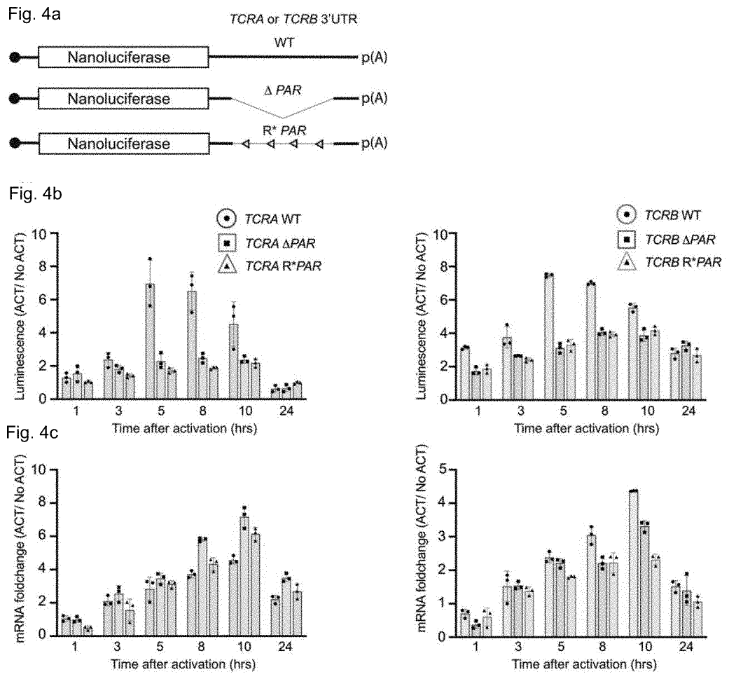

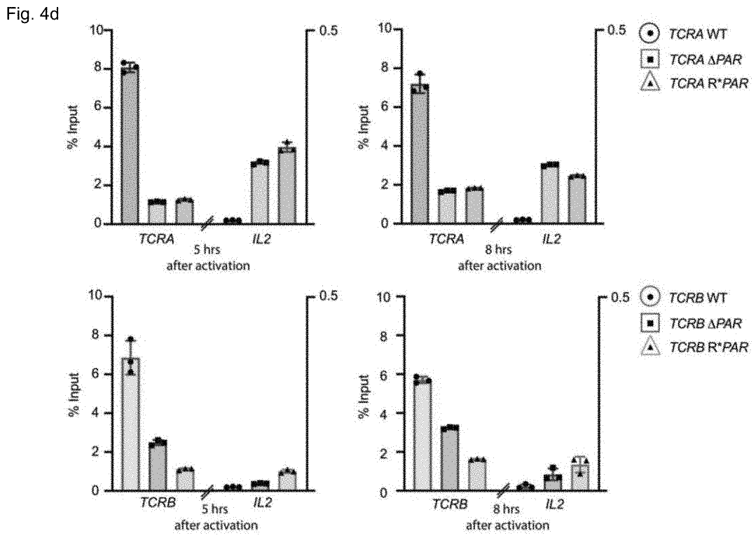

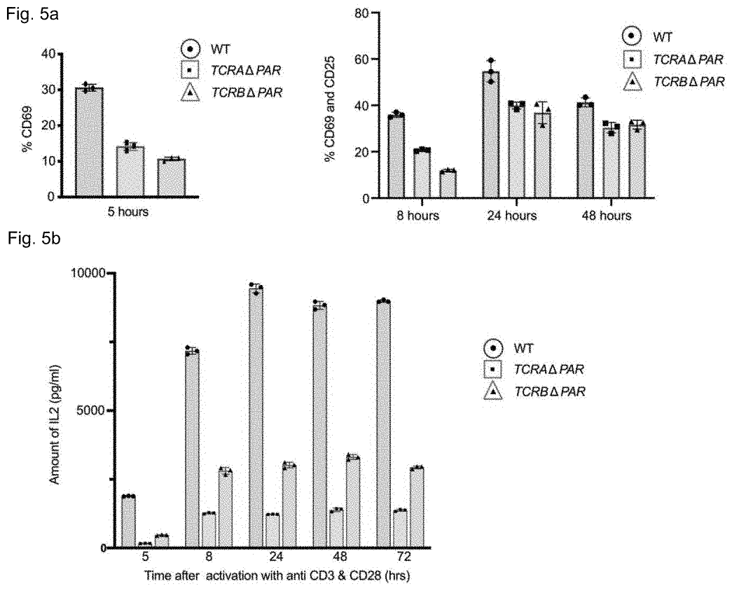

In order to understand the functional role of eIF3 interactions with the 3′-UTRs of the TCRA and TCRB mRNAs, we constructed nanoluciferase reporters with a 5′-UTR of the human beta globin gene (HBB) and a PEST destabilization domain. The PEST domain was first identified in rapidly degrading eukaryotic proteins through a literature survey. The PEST domain is rich in proline (P), glutamic acid (E), serine (S), and threonine residues (Rogers, 1986). The PEST domain reduces nanoluciferase protein half-life (Voon, 2005; Hall, 2012) and provides better time resolution of nanoluciferase expression after T cell activation. The reporter coding sequence (CDS) was then fused to the wild-type TCRA or TCRB mRNA 3′-UTR sequence (WT), or 3′-UTR with the eIF3 PAR-CLIP site deleted (ΔPAR), or 3′-UTR with the reversed sequence of the eIF3 PAR-CLIP site (R*PAR, i.e. 5′-3′ sequence reversed to the 3′-5′ direction) (FIG. 4a), and stably transduced into Jurkat cells using a lentiviral vector.

Jurkat cells expressing the reporters with the WT TCRA or TCRB mRNA 3′-UTR sequences had higher luminescence that peaked 5-8 hours after activation, while cells expressing nanoluciferase from reporters with a deletion or reversal of the eIF3 PAR-CLIP site sequence showed no apparent translation burst. These results recapitulate the 3′-UTR dependence of the translational burst for the endogenous TCRA and TCRB mRNAs . Also as observed for the TCRA ΔPAR and TCRB ΔPAR Jurkat cell lines, cells expressing reporters with either TCRA ΔPAR or R*PAR or TCRB ΔPAR or R*PAR 3′-UTRs had no significant defect in the nanoluciferase mRNA levels compared to reporters with the corresponding WT 3′-UTR sequences. Finally, immunoprecipitation of eIF3 followed by qRT-PCR quantification of nanoluciferase mRNA showed that less nanoluciferase mRNA was bound to eIF3 when the 3′-UTR PAR-CLIP site was either deleted or reversed, compared to nanoluciferase mRNAs carrying the WT TCRA or TCRB 3′-UTR. Taken together, the experiments with nanoluciferase reporters show that the TCRA and TCRB 3′-UTRs are necessary and sufficient to drive the translational burst seen after Jurkat cell activation, and correlates with eIF3 binding to the eIF3 PAR-CLIP sequences.

The eIF3-mediated burst in TCR expression occurred when Jurkat cells were fully activated through TCR and CD28 signaling via anti-CD3 and anti-CD28 antibodies. Interestingly, the CD28 costimulatory signal alone was sufficient to cause the transient burst in TCRA protein levels in the early time points after activation, indicating that the CD28 costimulatory pathway drives the transient burst in TCR protein expression in Jurkat cells. CD28 signaling involves multiple membrane-associated events (Boomer & Green (2010) Cold Spring Harb Perspect Biol 2: a002436). Notably, anti-CD28 stimulation was sufficient to induce a transient burst in nanoluciferase expression from reporters harboring the WT TCRA mRNA 3′-UTR, but only when the reporters were tethered to the membrane via a CD3 zeta-derived N-terminal sequence. This burst did not occur when reporters harboring the TCRA 3′-UTR lacked the eIF3 PAR-CLIP site (TCRA ΔPAR). Furthermore, activation using only anti-CD3 antibodies led to increased reporter expression, but the increase peaked at a later time and did not drop off as significantly at later time points. These data indicate that the transient burst in TCR expression likely requires not only specific interactions between eIF3 and the TCRA and TCRB 3′-UTRs but also membrane-proximal CD28 signaling, as CD3zeta is co-translationally inserted into the membrane (Call & Wucherpfennig (2005) Annu Rev Immunol 23: 101-125).

We tested signaling pathways downstream of CD2819 to determine if they are required for the transient burst in TCR expression. We first stimulated Jurkat cells with anti-CD3/anti-CD28 antibodies and inhibited protein kinase AKT with different small molecule inhibitors. Inhibiting AKT kinase had no effect on the early increase in TCR protein levels, but failed to show the drop-off in TCR levels after the peak seen at 5-8 hours in untreated controls. We also tested whether mTOR activity affects TCR expression at early time points, using the inhibitor Torin-1, and found that inhibiting mTOR had no effect on the transient burst in TCR levels. These results indicate that CD28-mediated signaling events required for the early burst in TCR expression are independent of AKT and mTOR activity, and that AKT may play a role in the subsequent drop-off in TCR levels.

Cells expressing nanoluciferase from reporters with a deletion or reversal of the eIF3 PAR-CLIP site sequence led to reduction of the luminescence signal compared to cells expressing nanoluciferase with the WT 3′-UTR sequences (FIG. 4b), indicating that eIF3 binding to the TCRA and TCRB 3′-UTR sequences is required for efficient translation. Furthermore, similar to the Jurkat cell lines with either TCRA or TCRB eIF3 PAR-CLIP site deleted (TCRA ΔPAR or TCRB ΔPAR cells), there was no significant defect in the nanoluciferase mRNA levels in the ΔPAR and R*PAR cells compared to cells with intact TCRA and TCRB 3′-UTR sequences, as measured by qRT-PCR (FIG. 4c). In fact nanoluciferase mRNA fold changes in the ΔPAR and R*PAR cells were either higher or equal to the levels in cells with reporters containing intact TCRA and TCRB 3′-UTR sequences at all time points after activation with anti-CD/anti-CD28 antibodies (FIG. 4d).

In order to test whether T cell activation is affected when the eIF3 PAR-CLIP site in the TCRA or TCRB 3′-UTR is deleted, we measured the kinetics of CD69 and CD25 activation marker expression by flow cytometry and IL2, a secreted cytokine, using ELISA after activating the cells with anti-CD3/anti-CD28 antibodies. In both TCRA ΔPAR and TCRB ΔPAR cell lines, less than 50% of WT CD69 levels was detected on the cell surface 5 hours after activation (FIG. 5a). After 8 hours of activation, when CD25 begins to appear on the cell surface, the TCRA ΔPAR and TCRB ΔPAR cells expressed low levels of CD69 and CD25 (FIG. 5a), and expression at later time points never reached that of WT cells (FIG. 5a). The expression levels of CD69 and CD25 mirror the western blot of TCRA levels, which were strongly affected at early time points after activation, and only increased moderately at later time points. Similar to the CD69 and CD25 cell surface markers, IL2 levels were significantly lower in activated TCRA ΔPAR and TCRB ΔPAR cells compared with WT cells, with the amounts barely increasing at later time points (FIG. 5b). This clearly showed decreased TCRA and TCRB protein expression levels due to deletion of the eIF3 PAR-CLIP sites in the TCRA or TCRB 3′-UTR decreased overall T cell activation at short and long time points.

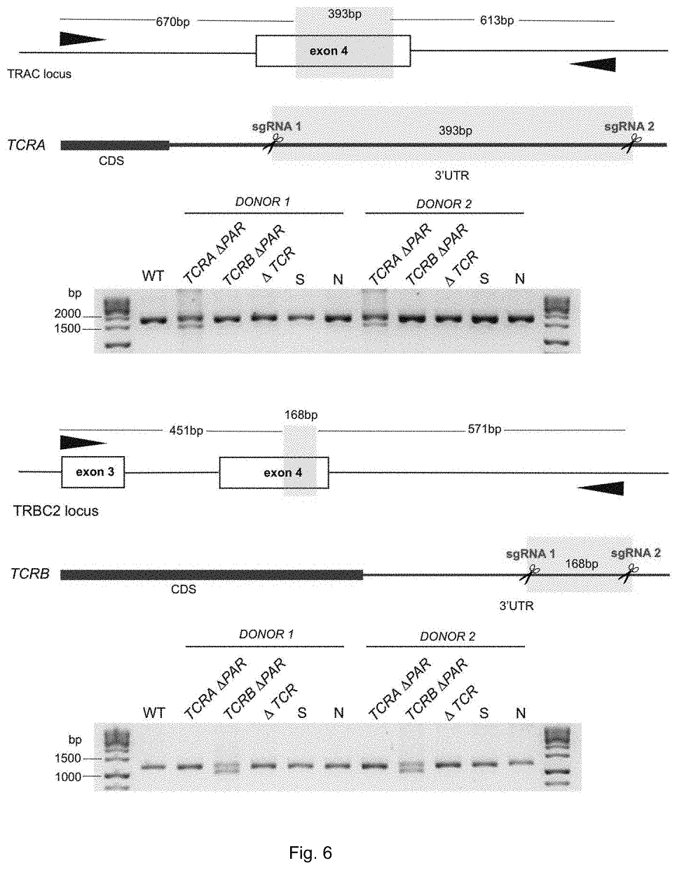

We next investigated whether the biochemical and phenotypic results obtained in TCRA ΔPAR and TCRB ΔPAR Jurkat cells could be replicated in primary human T cells. To delete the entire PAR-CLIP site in the 3′-UTR of TCRA and TCRB in primary human T cells, we electroporated T cells from two donors with gRNA/Cas9 RNPs harboring the same target sequences we previously used in Jurkat cells. PCR analysis (FIG. 6) showed approximately 50-60% of the cells had the deletion of the eIF3 PAR-CLIP site in the TCRA 3′-UTR or in the TCRB 3′-UTR (TCRA ΔPAR or TCRB ΔPAR, respectively). Since we were unable to obtain a clonal population of TCRA ΔPAR or TCRB ΔPAR primary T cells, we created additional edited cell lines from both donors as controls to better understand the phenotype of the ΔPAR cell lines. These control cells are: only one gRNA targeting TCRA 3′-UTR and TCRB 3′-UTR region, gRNA targeting the CDS of TCRA leading to complete knockout (KO) of the TCR, scrambled gRNA and cells that were mixed with the gRNA/Cas9 RNPs but not nucleofected.

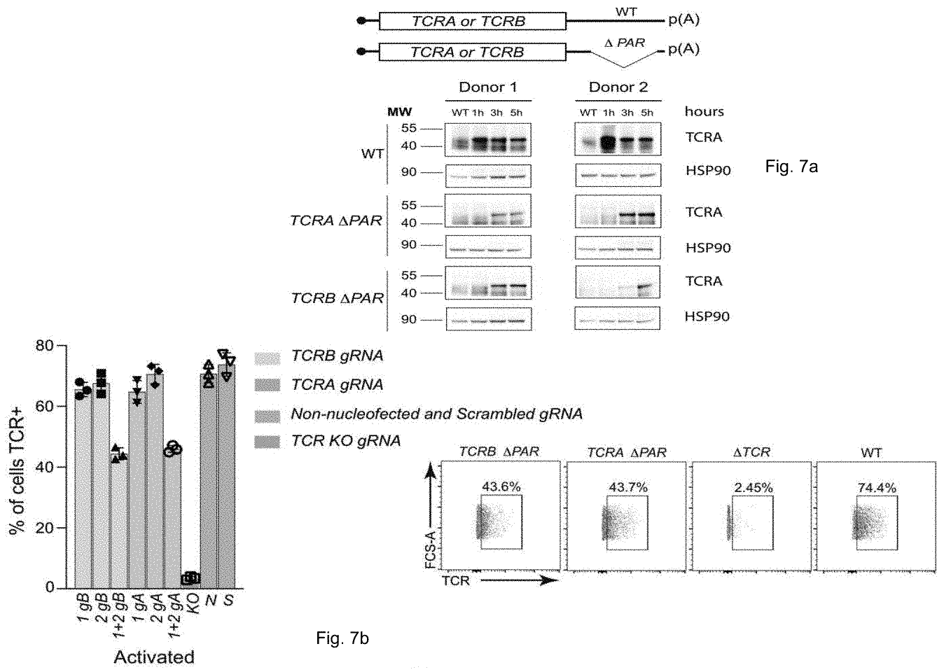

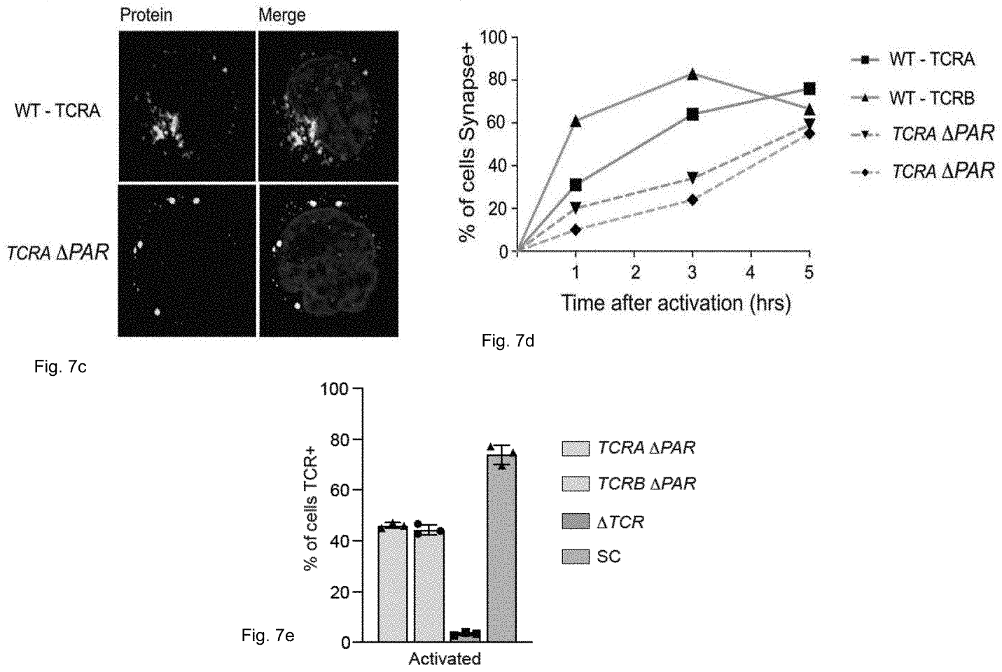

From the primary T cells edited as described above, we first measured total TCR protein levels of both TCRA ΔPAR and TCRB ΔPAR cells by western blot at different time points after stimulation with anti-CD3 and anti-CD28 antibodies (FIG. 7a). An anti-TCRA antibody was used to measure TCR levels in both TCRA ΔPAR and TCRB ΔPAR cells (Ohashi, 1985; Koning, 1988). In both TCRA ΔPAR and TCRB ΔPAR primary T cells, TCRA protein levels were clearly reduced or hardly present, especially at the early time points after stimulation compared to the WT cells. We also observed a very clear burst of TCRA protein levels upon activation in the WT cells, while in the TCRA ΔPAR and TCRB ΔPAR cells, this burst was not visible even when the protein levels increased at the later time points. We next stained cells with a fluorescently tagged anti-TCR antibody and used flow cytometric analysis to measure the percent of cells expressing TCR on the surface before and after activation (FIG. 7b). Compared to one-gRNA edited, scrambled-gRNA and non nucleofected cells, the ΔPAR cells (cells treated with both gRNAs targeting the eIF3 PAR-CLIP site) clearly expressed less TCR. However, TCR expression levels were not as low as seen with the complete TCR KO cells. This could be due to the incomplete editing of the PAR-CLIP sites in the T cell population, or possibly due regulation occurring in addition to that derived from the 3′-UTR elements.

Next we checked whether the reduced TCR protein levels affected immune synapse formation in the TCRA ΔPAR and TCRB ΔPAR cells, especially at earlier time points after activation (FIG. 7c). We stained TCRA or TCRB proteins in fixed cells with either anti-TCRA or anti-TCRB antibodies and used fluorescently tagged secondary antibody to image them with an epifluorescent microscope. To match the protein levels observed in western blots (FIG. 7a) with immune synapse formation, the same pool of activated cells were used for both assays. We counted a total of 100 cells for each time point after activation with anti-CD3 and anti-CD2S for both donors and noted the number of cells expressing either one or more synapses (FIG. 7d). An immune synapse was not observed when the cells were not activated. However, after 1 hour of activation, 30%-60% of the WT cells formed an immune synapse while only 10%-20% of the TCRA ΔPAR or TCRB ΔPAR cells formed a synapse. Furthermore, while up to 60%-80% of WT cells formed immune synapses at later time points, only 30%-50% of the TCRA ΔPAR and TCRB ΔPAR cells did so (FIG. 7d). We also stained cells with a fluorescently tagged anti-TCR antibody and used flow cytometric analysis to measure the levels of TCR on the cell surface with and without activation with I+PMA (FIG. 7c, 7e). A substantially lower percentage of the TCRA. ΔPAR and TCRB ΔPAR cell populations expressed TCR on the cell surface compared to SC cells or cells edited with a single gRNA (FIG. 7d, 7e). This supports the model that regulation of TCR protein synthesis is mediated by the TCRA and TCRB mRNA 3′-UTR elements that interact with eIF3 for robust TCR clustering leading clearly impact immune synapse formation.

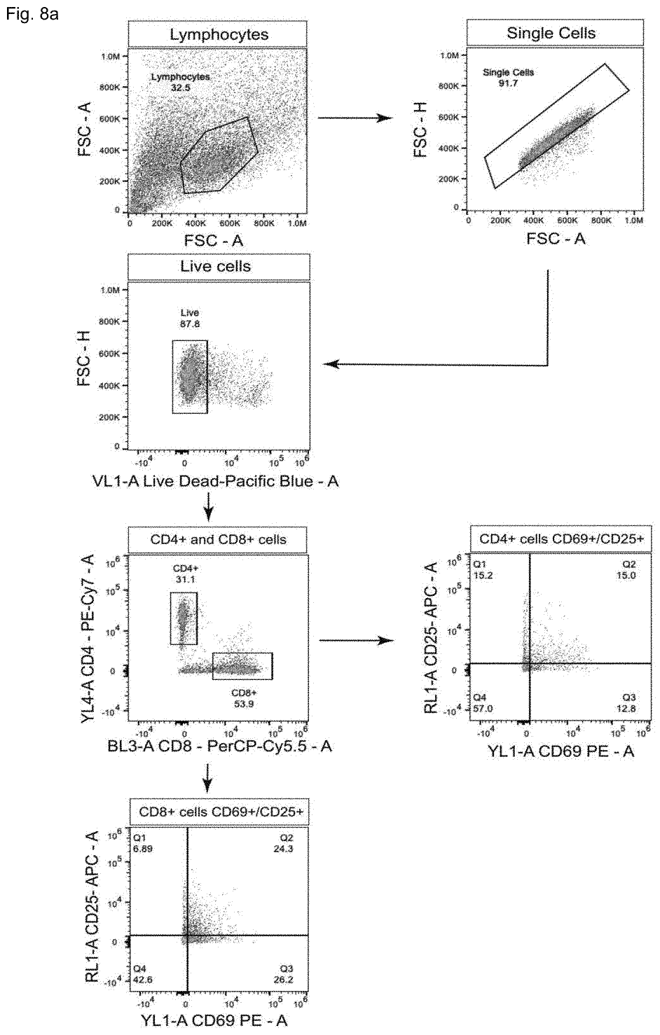

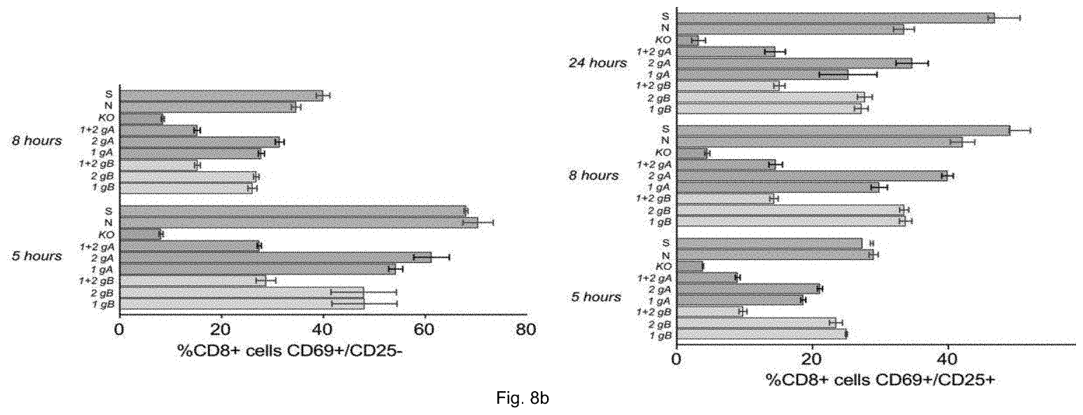

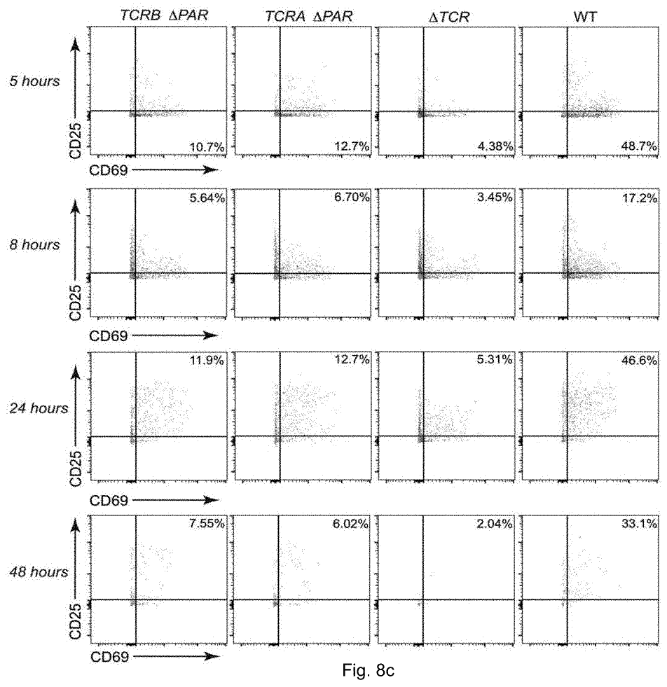

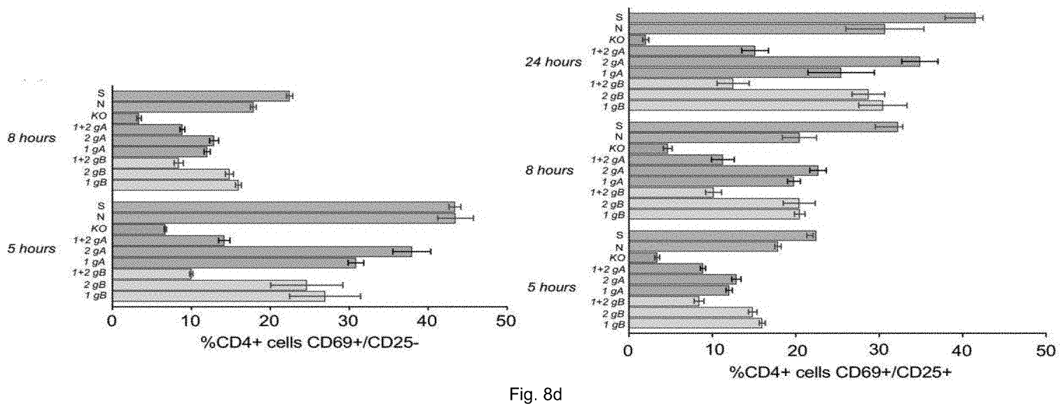

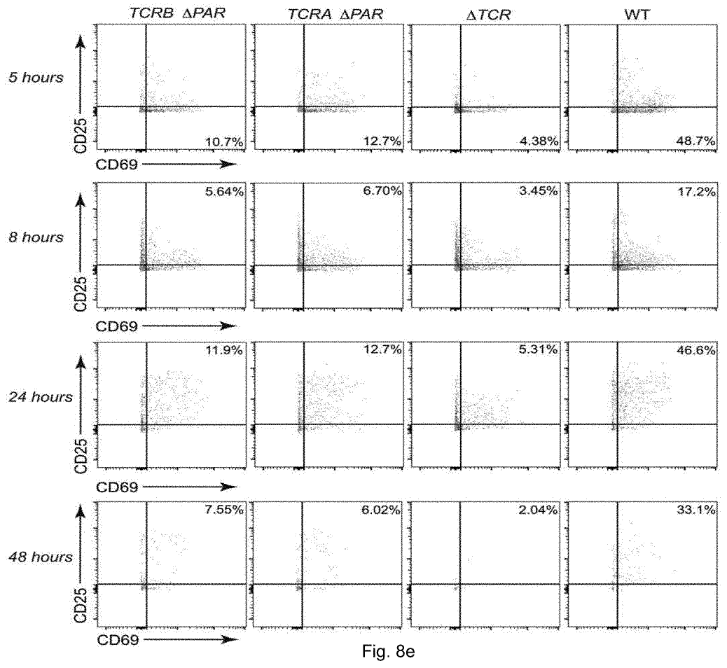

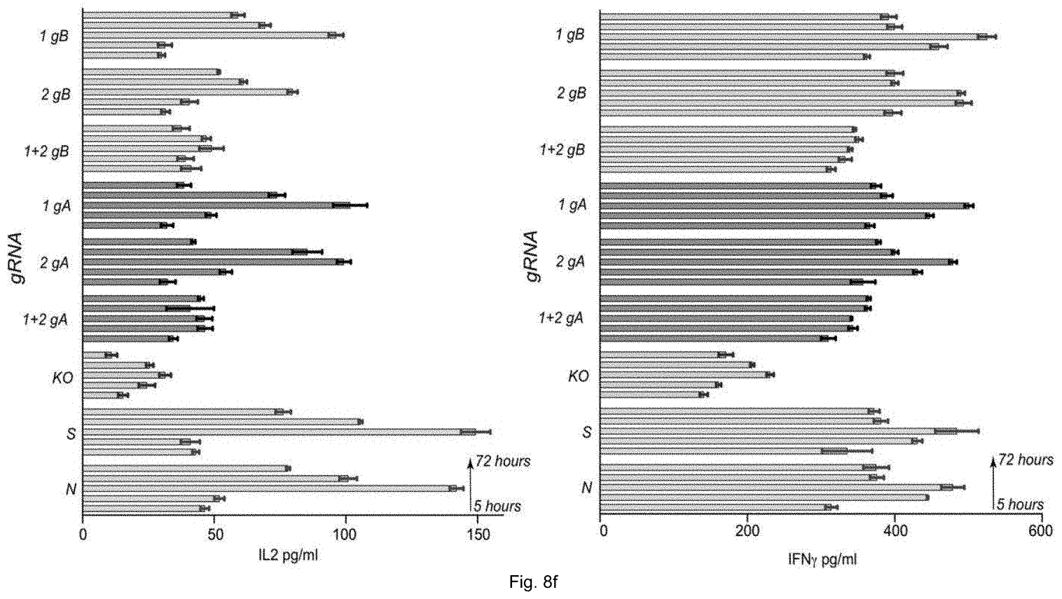

To test whether the defect in immune synapse formation in the TCRA ΔPAR and TCRB ΔPAR cells reflects general deficiency in T cell activation, we measured the T cell activation markers CD69 and CD25 by flow cytometry and secreted cytokines IL2 and IFNγ using ELISA after T cell activation with anti-CD3/anti-CD28 antibodies (FIG. 8). Both TCRA ΔPAR and TCRB ΔPAR CD4+ and CD8+ cells expressed less CD69 after 5 and 8 hours after activation, compared to either single-gRNA edited, scrambled-gRNA or non-nucleofected cells (FIG. 8b-e). At later time points after activation (8 hours, 24 hours and 48 hours) both TCRA ΔPAR and TCRB ΔPAR cells consistently expressed lower levels of both CD69 and CD25 activation markers and secreted lower amounts of IL2 and IFNγ cytokines, compared to the control cell lines (FIG. 8b-f). However, both TCRA ΔPAR and TCRB ΔPAR cells expressed higher amounts of CD69 at earlier time points and CD69, CD25, IL2 and IFNγ at later time points compared to the total TCR KO indicating that TCR protein expression is mediated by additional regulation pathways in addition to the eIF3 3′-UTR PAR-CLIP sites in TCRA and TCRB mRNAs. Taken together, the TCRA ΔPAR and TCRB ΔPAR primary T cells exhibited activation defects at early and later time points, and align with the TCR translation defects observed by both western blot analysis and detected by immune synapse formation, consistent with eIF3-mediated regulation of T cell activation through interactions with the TCRA and TCRB mRNA 3′-UTR PAR-CLIP sites. These results also indicate that eIF3-responsive mRNA 3′-UTR elements can be used to improve chimeric antigen receptor (CAR) expression and CAR-T cell responsiveness (Eyquem et al (2017) Nature 543:113-117; Watanabe et al (2018) Front Immunol 9:2486) as well as improving receptor expression of other engineered receptors in T cells and NK cells including TCRs and ACTRs.

T cells engineered to express chimeric antigen receptors (CARS) for cancer immunotherapy now in use clinically (Kalos et, al (2011) Sci Transl Med 3: 95ra73; Kochenderfer et al (2013) Blood 122:4129-4139; Park et al (2018) N. Engl J. Med. 378: 449-459) and in next-generation designs (Eyquem et al (2017) Nature 543: 113-117) express the CAR from stably integrated transgenes that employ artificial 3′-UTRs in the CAR-encoding mRNA. To test whether these 3′-UTRs induce a transient burst in translation as observed with the WT TCRA or TCRB 3′-UTRs, we fused 3′-UTR sequences now in use for CAR expression to nanoluciferase reporters and expressed these in Jurkat cells. In contrast to the TCRA 3′-UTR, the other 3′-UTR elements failed to induce the burst in nanoluciferase expression. These data indicate that fusing the TCRA or TCRB 3′-UTR sequences to engineered CAR genes can be used to obtain a more physiological expression pattern seen for the endogenous TCR.

Recent experiments indicate that T cells must cross a “threshold” of T cell receptor signaling to commit to activation and proliferation (Qu-Yeung et al (2014) Proc Natl Acad Sci USA 111: E679-688; Au-Young et al (2017) J. Immunol. 198: 2445-2456), setting up a “digital” response to antigen recognition (Au-Yeung, 2014; Au-Yeung, 2017, Allison et al (2016) Elife 5; Richard et al (2018) Nat. Immunol 19: 849-958). The response threshold involves integration of intensity and duration of TCR signaling (Au-Yeung, 2014; Au-Yeung, 2017; Allison, 2016; Richard, 2018) and spans a wide range of TCR antigen affinity (Au-Yeung, 2014; Au-Yeung, 2017; Richard, 2018). Notably, T cell commitment to clonal expansion and differentiation can occur with as little as 1 to 2 hours of TCR stimulation for effector CD4+ and naive CD8+ T cells (Iezzi & Lanzavecchia (1998) Immunity 8:89-95; van Stipdonk et al (2001) Nat. Immunol. 2:423-429). This time period spans the burst in TCR protein synthesis mediated by eIF3 interactions with the TCRA and TCRB 3′-UTR elements. Naive CD4+ T cells require a longer duration of TCR signaling of ˜20 hours (Iezzi, 1998; Schrum et al (2005) Eur. J. Immunol. 35:449-459). Although we were not able to distinguish levels of TCR translation in isolated CD8+ and CD4+ T cells, subsequent events in T cell activation including CD69 and CD25 expression, and IL2 and IFNγ secretion, were equally affected in CD8+ and CD4+ cells in which the 3′-UTR R PAR-CUP sites were deleted. In an immune response, CD28 engagement serves as the second signal required for T cell activation (Harding et al (1992) Nature 356: 607-609; Harding & Allison (1993) J. Exp. Med 177:1791-1796) and affects the first minutes of TCR-mediated signaling (Michel et al (2001) Immunity 15: 935-945; Green et al (2000) J. Immunol 164: 3591-3595; Green et al (1994) Immunity 1: 501-508; Tuosto & Acuto (1998) Eur. J. Immunol 28: 2131-2142; Shahinian et al (1993) Science 261: 609-612). Here we now show CD28-mediated signaling is also needed for the burst of TCR translation on the hour timescale. Taken together, our results indicate eIF3 controls TCRA and TCRB mRNA translation during the first critical hours after antigen recognition that lead to subsequent T cell commitment to proliferation and differentiation. Given that CD28 is required for PD-1 mediated inhibition of T cell activation (Kamphorst et al (2017) Science 355:1423-14:27; Hui et al (2017) Science 355: 1428-4433), it will be interesting to determine the role of eIF3-mediated control of TCR expression in PD-1 checkpoint blockade-based cancer immunotherapy (Jiang et al (2019) Hum. Vaccin. Immunother. 15:1111-1122). Additionally, our results using nanoluciferase reporters suggest that eIF3-responsive mRNA 3′-UTR elements could be used to improve chimeric antigen receptor expression and CAR-T cell responsiveness (Eyquem et al (2017) Nature 543: 113-117; Watanabe et al (2018) Front Immunol 9:2486).

The eIF3 PAR-CLIP experiment we presented here provided a snapshot of eIF3-RNA interactions that occur at the time TCR translation is most sensitive to eIF3 regulation (5 hours in Jurkat cells, FIG. 3). At this point in time, eIF3 crosslinks to multiple mRNAs encoding proteins involved in immune cell function (Table 1). Notably, additional layers of translation regulation also contribute to T cell function (Tan et al (2017) Immunity 46:488-503), particularly with respect to mTOR signaling (Miyamoto et al (2005) J Biol Chem 280:282.51-28264; Myers et al (2019) Immunol Rev 291:134-153), and carbon metabolism (Ricciardi et al (2018) Cell Metab 28:895-906.e5; Manfrini et al (2017) Dev Camp Immunol 77:69-76). It should now be possible to use the present PAR-CLIP experiments to elucidate additional roles for eIF3-mediated translation regulation and to more fully map the system-wide role of translation regulation in T cell activation.

### Conclusion

Although a preferred method of introducing the TCRA or TCRB 3′-UTR noncoding elements using Cas9 and dual sgRNAs is described, other methods of introducing engineered TCRs or CARs into immune cells are available. For example, those with ordinary skill in the art could use Cas12a enzymes (Jiang, 2018), CasX (Liu, 2019), or zinc-finger nucleases or TALENs (Khan, 2019).

Although the preferred noncoding sequences to introduce into the 3′-UTR of engineered TCRs or CARs are human sequences, one of ordinary skill in the art can obtain homologous sequences in other mammals using commonly-available sequence alignment programs such as megablast or discontiguous megablast (McGinnis, 2004). Using these sequence alignment programs, homologous sequences can be identified in mammals including Hominidae (Pongo, Pan, Gorilla, and Homo genera), Hylobatidae (lesser apes), Cercopithecidae (Old World monkeys), and more distantly-related mammals including Perissodactyla (Odd-toed ungulates), Artiodactyla (Even-toed ungulates), Carnivora including Canidae, and Chiroptera including bats. For example, one can conduct a discontiguous megablast search using the National Center for Biotechnology Information (NCBI) BLAST server ( ) and human chromosome 14 (sequence NC_000014.9) with nucleotide range 22,551,700-22,552,073 (see FIG. 2a), yielding homologous sequences from the above mammalian taxa with 83%-97% sequence coverage, and at least 74% sequence identity at the nucleotide level. As another example, one can conduct a discontiguous megablast search using the National Center for Biotechnology Information (NCBI) BLAST server (http://blast.ncbi.nlm.nih.gov/Blast.cgi) and human chromosome 7 (sequence NC_000007.14) with nucleotide range 142,802,561-142,802,695 (See FIG. 2a), yielding homologous sequences from the above mammalian taxa with 93%-100% sequence coverage, and at least 90% sequence identity at the nucleotide level. Materials and Methods

| TABLE 3 | |
|---|---|
| Guide RNA Sequences | |
| Name | Sequence |
| TCRA sgRNA1, | GAACUCUCCUACCCCCAAGG |
| TCRA crRNA1 | (SEQ ID NO: 10) |
| TCRA sgRNA2, | GGGAAAUUAUCUCAUUAUCG |
| TCRA crRNA2 | (SEQ ID NO: 11) |
| TCRB sgRNA1, | GUGAGGAUGAAGAAUGACCU |
| TCRB crRNA1 | (SEQ ID NO: 12) |
| TCRB sgRNA2, | UUAGCCUAUUUCGUACUUGG |
| TCRB crRNA2 | (SEQ ID NO: 13) |

TABLE 4
Primer Sequences

| Name | Sequence |
|---|---|
| TRAC_qPCR_FW1 | TGTCAGTGATTGGGTTCCGA |
| | (SEQ ID NO: 14) |
| TRAC_qPCR_RV1 | CAGATCTCAGCTGGACCACA |
| | (SEQ ID NO: 15) |
| TRBC2_qPCR_FW1 | CTCCAGATACTGCCTGAGCA |
| | (SEQ ID NO: 16) |
| TRBC2_qPCR_RV1 | CTATCCTGGGTCCACTCGTC |
| | (SEQ ID NO: 17) |
| NLucP_FW1 | TCCCGTATGAAGGTCTGAGC |
| | (SEQ ID NO: 18) |
| NLucP_RV1 | TCATCCACAGGGTACACCAC |
| | (SEQ ID NO: 19) |
| GFP_FW_1 | TACTTCTCGATGCGGGTGTT |
| | (SEQ ID NO: 20) |
| GFP_RV_1 | TCTACCACTTCGGCACCTAC |
| | (SEQ ID NO: 21) |

TABLE 5
Antibodies

| Name | Company | Cat # | Clone # |
|---|---|---|---|
| anti-CD69 | BioLegend | 310906 | FN50 |
| anti-CD25 | BioLegend | 3402610 | BC96 |
| anti-TCRA | Santa Cruz Biotechnology | SC-515719 | |
| anti-HSP90 | Santa Cruz Biotechnology | SC-69703 | |

Jurkat Cell Culture

Human Jurkat, Clone E6-1 (ATCC TIB-152) was purchased from American Type Culture Collection (ATCC) and was maintained in RPMI 1640 Medium (ATCC modification) with 10% FBS (VWR Life Science Seradigm) and 0.01% Penicillin-Streptomycin (10,000 U/mL). The cells were maintained between 1 million to 8 million cells/mL. When cells were stimulated they were always maintained at 8 million cells/mL.

Isolation of Human Primary T Cells

Primary human T cells were isolated from healthy human donors from leukoreduction chambers after Trima Apheresis (Blood Centers of the Pacific). Peripheral blood mononuclear cells (PBMCs) were isolated from whole blood samples by Ficoll centrifugation using SepMate tubes (STEMCELL, per manufacturer's instructions). T cells were isolated from PBMCs from all cell sources by magnetic negative selection using an EasySep Human T Cell Isolation Kit (STEMCELL, per manufacturer's instructions). Unless otherwise noted, isolated T cells were stimulated as described below and used directly (fresh).

Primary Human T cell Culture

Bulk T cells were cultured in XVivo15 medium (STEMCELL) with 5% fetal bovine serum (FBS), 50 μM 2-mercaptoethanol, and 10 μM N-acetyl-cystine Immediately after isolation, T cells were stimulated for 2 days with anti-human CD3/CD28 magnetic dynabeads (ThermoFisher) at a bead to cell concentration of 1:1, along with a cytokine cocktail of IL-2 at 200 U/mL (UCSF Pharmacy). After electroporation, T cells were cultured in media with IL-2 at 500 U/mL. Throughout the culture period T cells were maintained at an approximate density of 1 million cells per mL of media. Every 2-3 days after electroporation, additional media was added, along with additional fresh IL-2 to bring the final concentration to 500 U/mL, and the cells were transferred to larger culture flasks as necessary to maintain a density of 1 million cells per mL.

After 9 days of culturing, edited primary T cells were transferred to fresh media lacking IL2. The T cells were then stimulated with anti-CD3 and anti-CD28 antibodies using flat bottom plates coated with anti-CD3 antibody (at a 10 μg/mL concentration), and anti-CD28 antibody added to the cell culture media at a concentration of 5 μg/mL.

Cell Stimulation

The Jurkat cells used for the for the PAR-CLIP experiment were stimulated with 1× Cell Stimulation Cocktail, containing phorbol 12-myristate 13-acetate (PMA) and ionomycin (Catalog number: 00-4970-93).

Unless otherwise stated all other Jurkat cell simulations were performed with anti-CD3 and anti-CD28 antibodies. Flat bottom plates were coated with anti-CD3 antibody (at a 10 μg/mL concentration), and anti-CD28 antibody was added to the cell culture media at a concentration of 5 μg/mL.

4-thiouridine optimization Jurkat cells were seeded, so that they reached 8×10⁵ cells ml⁻¹ seeding density on the day of the experiment. Varying concentrations of s4U were added to the cells (50 uM, 75 uM, 100 uM, or none as a negative control). The cells were then incubated for different time points: 8 hours, 10 hours, 12 hours, or 16 hours. After each incubation time cell viability was determined using the CellTiter-Glo assay (Promega), according to the manufacturer's instructions. Concentrations at which the relative luminescence in the presence of s4U (luminescence of the s4U treated cells/luminescence of the untreated cells) exceeded 95% were considered non-toxic. Based on these measurements, we used 50 μM of 4-thiouridine for PAR-CLIP experiments.

PAR-CLIP

Two biological replicates were used to perform PAR-CLIP analysis as described in (Lee, 2015), with modifications for Jurkat cells. A total of 55 million Jurkat cells seeded at 8 million cells/mL were treated with 50 μM of 4-thiouridine (Sigma) 7 hours, then stimulated with 1× Cell Stimulation cocktail for 5 hours in media containing 50 μM of 4-thiouridine (FIG. 1a). The same number of cells were treated with 50 μM of 4-thiouridine (Sigma) for 12 hours without stimulation as a non-activated control. The cells were then crosslinked on ice with 365 nm UV irradiation at an energy of 0.2 J cm⁻². The cells were pelleted by centrifugation at 100×g for 15 min at 4° C., and the pellet was resuspended in three volumes of NP40 lysis buffer (50 mM HEPES-KOH pH 7.5, 150 mM KCl, 2 mM EDTA, 0.5% Nonidet P-40 alternative, 0.5 mM dithiothreitol (DTT), 1 Complete Mini EDTA-free Protease Inhibitor Cocktail tablet (Roche)). The cell suspension was incubated on ice for 10 mM, passed through an 18G needle five times, and centrifuged at 13,000×g for 15 mM at 4° C. and RNAs were lightly digested by treatment with MNase (Thermo Scientific) at a final concentration of 0.05 U/μL for 20 mM at 16° C. For each PAR-CLIP assay 1000 μL of Dynabeads (Invitrogen) and 800 μL of anti-EIF3B antibody (Bethyl A301-761A) were used. The remaining steps of the PAR-CLIP analysis was performed exactly as described in (Danan, 2016; Lee, 2015) with the exception of using MNase at 5 U/μL for the on-bead digestion step.

PAR-CLIP Computational Analysis

PAR-CLIP cDNA libraries were sequenced on an Illumina HiSeq 2500 platform. Clusters of overlapping sequence reads mapped against the human genome version hg38 were generated using the PARalyzer software (Corcoran, 2011) incorporated into a pipeline (PARpipe; haps://ohlerlab.mdc-berlin.de/software/PARpipe_119/) with default settings. Binding sites were categorized using the Gencode GRCh38.p13 GTF annotations, https://www.gencodegenes.org/human/.

The default PARpipe settings are:

Conversion=T>C

Minimum read count per group=5

Minimum read count per cluster=5

Minimum read count for kde=3

Minimum cluster size=11

Minimum conversion locations for cluster=2

Minimum conversion count for cluster=2

Minimum read count for cluster inclusion=1

Minimum read length=20

Maximum number of non conversion mismatches=1

To eliminate the PCR biases of the library a random bar code was introduced into the 3′ adapter and all the reads that matched the random barcode were collapsed into single reads.

Comparison of eIF3 PAR-CLIP Results in Jurkat and HEK293T Cells

To compare RNAs crosslinked to eIF3 in HEK293T cells (Lee 2015, ibid) with those in activated Jurkat cells, we selected the top genes in the PAR-CLIP lists in Jurkat cells sorted by read count from each library. We used the same number of candidate genes as were identified in HEK293T cells, categorized by eIF3 subunit crosslinked to the RNA.

PAR-CLIP Pathway analysis To determine biological pathways enriched in the set of mRNAs that crosslinked to eIF3 in activated Jurkat cells, we used genes with at least 100 total aligned reads, as determined in the PARpipe analysis described above (Mukherjee (2019) Nucl Acid Res 47:570-581), from the EIF3A/C/B samples. Since PAR-CLIP reads are short, it is not possible to determine with certainty which mRNA transcript isoform cross-linked with eIF3. We therefore chose the most abundant mRNA transcript isoform for each gene, as determined by transcriptome profiling using kallisto (protein_coding category) (Bray et al (2016) Nat Biotechnol 34:525-527), as described in the Transcriptome Profiling section. Even with this choice, eIF3 crosslinks to mRNAs do not correlate with mRNA abundance. We used human genome GRCh38.p13 annotation to extract mRNA lengths by 5′-UTR, coding region and 3′-UTR (Ensembl Biomart) (Cunningham (2019) Nucl Acid Res 47:D745-D751). We then sorted these genes by the density of PAR-CLIP reads in the mRNA 5′-UTR region, prior to mapping pathways of transcripts that cross-linked to eIF3. Due the complexity of TCRA and TCRB transcript annotation, these transcripts were excluded from the normalization calculation, but included in the pathway analysis. We used the top 500 genes from the resulting EIF3A/C/B PAR-CLIP gene lists, sorted as described above, to analyze gene enrichment profiles in the STRING database (Szklarczyk et al (2019) Nucl Acid Res 47:D607-D613) The top tissue-specific categories involve the immune system and T cell function (Table 1). Note that the STRING database does not include TCR subunits in its analysis.

Metagene Analysis

We used the PAR-CLIP genes sorted as described above in the “PAR-CLIP pathway analysis” and mapped against the most abundant mRNA transcript isoforms to generate cumulative plots of the reads. Reads for the Jurkat TCRA and TCRB mRNAs were extracted from the unaligned PAR-CLIP reads using Bowtie version 1.0.0. These reads were combined with the mapped reads in the *.read.csv files generated by Parpipe. The combined reads were then sorted to extract only reads annotated as 5′-UTR, start codon, coding, stop codon, and 3′-UTR. We used the most abundant transcript isoform, as identified in the transcriptome profiling using kallisto (described below). Reads mapped to the 5′-UTR and start codon were normalized by the length of the 5′-UTR. Reads mapped to the coding region and stop codon were normalized by the length of the coding region. Finally, reads mapped to the 3′-UTR were normalized to the length of the 3′-UTR.

Transcriptome Profiling

RNA samples were extracted from HEK293T cells, non-activated Jurkat cells, and Jurkat cells activated for 5 hr with I+PMA, using the Direct-zol RNA Miniprep kit (Zymoresearch). The libraries were prepared using TruSeq Stranded RNA LT Ribo-Zero Gold kit (Illumina) following the manufacturer's instructions, with two biological replicates per condition. We used Cutadapt v.2.645 with a minimum read length of 20, 5′ end with a cutoff of 15 and the 3′ end with a cutoff of 10 in paired-end mode. RNA-seq reads were pseudoaligned using kallisto v.0.46.0 run in quant mode with default parameters to estimate transcript abundance (transcripts per million, TPM, (Bray 2016, ibid). The transcript index for kallisto was made with default parameters and GENCODE Release 32 (GRCh38.p13) FASTA file (Frankish (2019) Nucl Acids Res 47: D766-D773) and was run in quant mode with default parameters.

To compare HEK293T or non-activated Jurkat mRNA abundance with that in activated Jurkat cells, we used the output from kallisto for analysis using DeSeq2.47 We extracted all protein-coding transcripts (protein_coding) for the comparisons, and used tximport48 as part of the Bioconductor environment in R. We used all genes with ≥50 reads summed across all four samples in a comparison (two biological replicates per condition). Prior to making MA plots, we used the Approximate Posterior Estimation for generalized linear model (apeglm) shrinkage estimator for log fold changes (Zhu et al (2019) Bioinformatics 35:2084-2092).

Ribosome Profiling

Jurkat cells were seeded, and reached a density of 8×10⁵ cells ml−1 on the day of the experiment. The cells were then activated for 5 hr with I+PMA or left non-activated, in biological duplicate, rinsed with phosphate-buffered saline (PBS) containing cycloheximide (100 μg/mL) and triturated in lysis buffer (20 mM Tris-Cl pH 7.4, 150 mM NaCl, 5 mM MgCl2, 1 mM DTT, 100 μg ml−1 cycloheximide, 1% Triton X-100 and 25 U ml−1 DNAse I, Promega). The lysates were aliquoted, flash frozen and stored at −80° C. until used for ribosome footprint library preparation. Ribosomes and libraries were prepared as previously described (Ingolia et al (2012) Nat Protoc 7:1534-1550).

Mass Spectrometry

To identify eIF3 subunits that crosslinked with RNAs in the PAR-CLIP experiments, a portion of eIF3 immunoprecipitated using Dynabeads as described above were treated with nonradioactive ATP during the T4 PNK labeling step. The nonradioactive samples were then run on the same gel next to the radiolabeled PAR-CLIP samples, Coomassie stained (Pierce) and the bands that matched with the phosphorimager printout were excised from the gel and submitted for identification using one-dimensional LC-MS/MS.

sgRNA/Cas9 RNP Production

The sgRNA/Cas9 RNPs used to edit Jurkat cells were produced by complexing sgRNA (Synthego) to Cas9 as described in (Schumann, 2015) while RNPs to edit Primary Human T cells were produced by complexing a two-component gRNA (crRNA and tracrRNA, Dharmacon) to Cas9 as described in (Roth, 2019). The targeting sequences for the sgRNAs and crRNA are given in Table 1. Recombinant Cas9-NLS was obtained from MacroLab in the California Institute for Quantitative Biosciences.

Jurkat and Primary T Cell Electroporation

Jurkat cells used for electroporation were collected at passage 5 or lower and were maintained at a seeding density of 8 million cells/mL or lower. Primary T cells were isolated as described above. Prior to electroporation the cells were stimulated with magnetic anti-CD3/anti-CD28 Dynabeads (Thermofisher) for 48 hours. After 48 hours these beads were removed from the cells by placing cells on an EasySep cell separation magnet for 2 mM before electroporation. One million Jurkat or primary T cells cells were rinsed with PBS and then resuspended in 20 μL of Lonza electroporation buffer P3. The cells were then mixed with 2.5 μL Cas9 RNPs (50 pmol total) along with 2 μL of a 127-nucleotide non-specific single-stranded DNA oligonucleotide at 2 μg/μL (4 μg ssDNA oligonucleotide total). The cells were then electroporated per well using a Lonza 4D 96-well electroporation system with pulse code EH115 Immediately after electroporation, 80 μL of pre-warmed media (without cytokines) was added to each well, and the cells were allowed to rest for 15 min at 37° C. in a cell culture incubator while remaining in electroporation cuvettes. After 15 min, cells were moved to final culture flasks.

Western Blot

Western blot analysis was performed using the following antibodies: anti-TCRA (SC-515719), anti-HSP90 (SC-69703) (Table 3).

Total mRNA Isolation and Quantitative RT-PCR Analysis

Total RNA was isolated from whole cells for qRT-PCR using Quick RNA miniprep plus kit from Zymo Research following the manufacturer's instructions. Quantitative RT-PCR analysis was performed using the Power SYBR Green RNA-to-Ct 1-Step kit (Applied Biosystems) according to the manufacturer's instructions, and the QuantStudio™ 3 Real-Time PCR System (ThermoFisher). Each target mRNA was quantified in two biological replicates, with each biological replicate having three technical replicates.

Plasmids

The TCRA 3′-UTR and TCRB 3′-UTR sequences were amplified from Jurkat genomic DNA. The nanoluciferease sequence fused to a PEST domain was amplified from pNL1.2[NlucP] Vector Sequence (Promega) and was cloned into a modified CD813A vector (System Biosciences) containing the beta-globin (HBB) 5′-UTR using the In-Fusion® HD Cloning Kit (Takara). The subsequent mutations in the TCRA and TCRB 3′-UTRs were generated using these initial constructs.

The TCRA 3′-UTR and TCRB 3′-UTR sequences were amplified from Jurkat genomic DNA. For TCRA ΔPAR constructs, nucleotides 102-338 in the 3′-UTR of TCRA mRNA were deleted. For TCRB ΔPAR constructs, nucleotides 16-161 in the 3′-UTR of TCRB mRNA were deleted. TCRA/TCRB ΔPAR, TCRA/TCRB R*PAR, 3′-LTR (3′-Long Terminal Repeat) and bpA (bovine growth hormone polyadenylation signal) sequences were purchased as gblocks from IDT and were cloned into this plasmid backbone. The WPRE 3′-UTR sequence was amplified from the CD813A-1 (System Biosciences) vector.

For nanoluciferase reporters designed to be membrane-tethered, we fused the N-terminal sequence of CD3-zeta spanning the transmembrane helix (amino acids 1-60) to the nanoluciferase sequence above. To prevent interaction of the CD3-zeta-nanoluciferase fusion protein with the TCR, we made mutations in the CD3-zeta derived transmembrane helix that would disrupt interactions with the TCR, based on the cryo-EM structure of the complex⁶⁵ (PDB entry 6JXR) and consistent with earlier biochemical results66. The two mutations, L35F and D36V, are predicted to introduce a steric clash and disrupt an intra-membrane salt bridge, respectively, with other subunits in the TCR holo-complex. These CD3-zeta-nanoluciferase chimeras were cloned into the modified CD813A plasmids described above.

Generation of Stable Jurkat Cell Lines Expressing Nanoluciferase Reporter

For lentiviral production HEK293T cells were plated at a density of 80% in T-75 flasks the night before transfection. The cells were then transfected with plasmids: expressing the nanoluciferase, PsPAX2 and pCMV-VSV-G using the Lipofectamine 3000 reagent following manufacturer's instructions (ThermoFisher). Forty-eight hours after transfection, the viral supernatant was collected, filtered and concentrated using PEG-it Virus Precipitation Solution (System Biosciences) following manufacturer's instructions. The virus pellets were then resuspended in RPMI-1640 media and stored in −80° C.

The Jurkat cell transfections were done with multiple viral titers using TransDux™ MAX Lentivirus Transduction Reagent (System Biosciences) following the manufacturer's instructions. To test the viral transduction efficiency of the cells, forty-eight hours after viral transduction the % of cells expressing GFP was measured by FACS analysis and cells expressing less than 15% GFP was treated with 1 ug/ml Puromycin to the media. The cells were maintained in media with 1 ug/ml Puromycin but transferred into media without Puromycin the day before the cells were used for assays.

Luciferase Reporter Assay

The stable cell lines expressing the Nanoluciferease reporter were stimulated with anti CD3 and anti CD28 antibodies and the nanoluciferase activity was assayed after 1 hr, 3 hr, 5 hr, 8 hr, 10 hr and 24 hrs after stimulation using Nano-Glo® Luciferase Assay System (Promega). For each time point 200,000 cells were tested in triplicate for each cell line.

RNA Immunoprecipitation and qPCR

The EIF3B-RNA immunoprecipitations were done following exact same conditions used for the PAR-CLIP analysis with following changes. For each immunoprecipitation cell lysates were prepared in NP40 lysis buffer (50 mM HEPES-KOH pH 7.5, 150 mM KCl, 2 mM EDTA, 0.5% Nonidet P-40 alternative, 0.5 mM DTT, 1 Complete EDTA-free Proteinase Inhibitor Cocktail tablet per 50 mL of buffer) with 4 million cells. The lysates were then incubated with 50 μL Protein G Dynabeads conjugated with 20 μL of anti-EIF3B antibody (Bethyl A301-761A) for two hours at 4° C. After incubation the flow through was removed and the beads were washed 3× with 1 ml NP40 lysis buffer for each wash. The beads were then resuspended in 400 μL TRIzol reagent and vortexed for 1 minute. The RNA was extracted per TRIzol reagent manufacturers instructions and qPCR was performed as mentioned before using primers listed in Table 2.

Flow Cytometry and Cell Sorting.

Flow cytometric analysis was performed on an Attune NxT Acoustic Focusing Cytometer (ThermoFisher). Surface staining for flow cytometry and cell sorting was performed by pelleting cells and resuspending in 50 μL of FACS buffer (2% FBS in PBS) with antibodies at the indicated concentrations (Table 3) for 20 min at 4° C. in the dark. Cells were washed twice in FACS buffer before resuspension.

ELISA

The cell suspensions were collected after each time point of activation with anti-CD3/anti-CD28 for WT, TCRA ΔPAR or TCRB ΔPAR cells. The amount of secreted IL2 in the cell suspensions after activation anti-CD3/anti-CD28 for WT, TCRA ΔPAR or TCRB ΔPAR cells were measured by ELISA MAX™ Deluxe Set Human IL-2 (BioLegend) according to manufacturer's instructions.

RNA-FISH and Immunofluorescence

Jurkat cells were washed with PBS, fixed with 3.7% formaldehyde for 10 min at RT and washed twice for 5 min with PBS. PBS was discarded and 1 mL 70% ethanol was added. The cells were incubated at 4 C for 16 hours. The 70% ethanol was aspirated and the cells were washed once with 0.5 mL Stellaris RNA wash buffer A. The cells were then incubated with 100 μl Stellaris hybridization buffer containing Stellaris RNA FISH probe (Quasar 670 Dye, 125 nM) and anti-TCRA antibody (SC-515719 at 1:1000) for 16 hours at 28C. The cells were then washed twice with 0.5 mL Stellaris RNA wash buffer A containing anti-mouse secondary antibody conjugated with Alexa Fluor® 488 (Thermofisher) for 30 minutes at 37 C in the dark. In the second Stellaris RNA wash buffer A also contained DAPI. Finally the cells were washed once with 0.5 mL Stellaris RNA wash buffer B and mounted with mounting solution (Invitrogen). To capture the immune synapse at high resolution confocal ZEISS LSM 880 with Airyscan super-resolution mode with A Plan-Apochromat 63×/1.4 Oil objective (Zeiss) was used. To count cells containing immune synapse a Revolve Epi-Fluorescence microscope by Echo was used with a A Plan-Apochromat 40× objective (Olympus).

Bibliography

Almåsbak H, et al. Transiently redirected T cells for adoptive transfer. Cytotherapy. 2011 May; 13(5):629-40. Epub 2010 Dec. 21. PubMed PMID: 21174490.

Almasbak H, et al.. Inclusion of an IgG1-Fc spacer abrogates efficacy of CD19 CAR T cells in a xenograft mouse model. Gene Ther. 2015 May; 22(5):391-403. Epub 2015 Feb. 5. PubMed PMID: 25652098.

Bernareggi D, Pouyanfard S, Kaufman DS. Development of innate immune cells from human pluripotent stem cells. Exp Hematol. 2019 March; 71:13-23. Epub 2019 Jan. 4. PubMed PMID: 30611869; PubMed Central PMCID: PMC6401218.

Corcoran D L, Georgiev S, Mukherjee N, Gottwein E, Skalsky R L, Keene J D, Ohler U. PARalyzer: definition of RNA binding sites from PAR-CLIP short-read sequence data. Genome Biol. 2011 Aug. 18; 12(8):R79. PubMed PMID: 21851591; PubMed Central PMCID: PMC3302668.

Crews C M. Targeting the undruggable proteome: the small molecules of my dreams. Chem Biol. 2010 Jun. 25; 17(6):551-5. Review. PubMed PMID: 20609404; PubMed Central PMCID: PMC2925121.

Danan C, Manickavel S, Hafner M. PAR-CLIP: A Method for Transcriptome-Wide Identification of RNA Binding Protein Interaction Sites. Methods Mol Biol. 2016; 1358:153-73. PubMed PMID: 26463383; PubMed Central PMCID: PMC5142217.

Dang C V, Reddy E P, Shokat K M, Soucek L. Drugging the ‘undruggable’ cancer targets. Nat Rev Cancer. 2017 Jun. 23. [Epub ahead of print] Review. PubMed PMID: 28643779.

Disney M D. Inhibiting Translation One Protein at a Time. Trends Biochem Sci. 2017 June; 42(6):412-413. Epub 2017 May 15. PubMed PMID: 28522328.

Eyquem J, et al. Targeting a CAR to the TRAC locus with CRISPR/Cas9 enhances tumour rejection. Nature. 2017 Mar. 2; 543(7643):113-117. Epub 2017 Feb. 22. PubMed PMID: 28225754; PubMed Central PMCID: PMC5558614.

Ferreira L M R, Muller Y D, Bluestone J A, Tang Q. Next-generation regulatory T cell therapy. Nat Rev Drug Discov. 2019 October; 18(10):749-769. doi: 10.1038/s41573-019-0041-4. Epub 2019 Sep. 20. Review. PubMed PMID: 31541224.

Galán A, Comor L, Horvatić A, Kuleě J, Guillemin N, Mrljak V, Bhide M. Library-based display technologies: where do we stand? Mol Biosyst. 2016 Jul. 19; 12(8):2342-58. doi: 10.1039/c6mb00219f. Review. PubMed PMID: 27306919.

Hall M P, et al. Engineered luciferase reporter from a deep sea shrimp utilizing a novel imidazopyrazinone substrate. ACS Chem Biol. 2012 Nov. 16; 7(11):1848-57. doi: 10.1021/cb3002478. Epub 2012 Aug 30. PubMed PMID: 22894855; PubMed Central PMCID: PMC3501149.

Hanes J, Jermutus L, Plückthun A. Selecting and evolving functional proteins in vitro by ribosome display. Methods Enzymol. 2000; 328:404-30. PubMed PMID: 11075357.

Haque M, et al. Stem cell-derived tissue-associated regulatory T cells ameliorate the development of autoimmunity. Sci Rep. 2016 Feb. 5; 6:20588. doi: 10.1038/srep20588.

Huppertz I, et al. iCLIP: protein-RNA interactions at nucleotide resolution. Methods. 2014 February; 65(3):274-87. doi: 10.1016/j.ymeth.2013.10.011. Epub 2013 Oct. 25. PubMed PMID: 24184352; PubMed Central PMCID: PMC3988997.

Jensen M C, Riddell S R. Design and implementation of adoptive therapy with chimeric antigen receptor-modified T cells. Immunol Rev. 2014 January; 257(1):127-44. doi: 10.1111/imr.12139. Review. Erratum in: Immunol Rev. 2014 March; 258(1):259. PubMed PMID: 24329794; PubMed Central PMCID: PMC3991306.

Jiang D J, Xu C L, Tsang S H. Revolution in Gene Medicine Therapy and Genome Surgery. Genes (Basel). 2018 Nov. 26; 9(12). pii: E575. doi: 10.3390/genes9120575. Review. PubMed PMID: 30486314; PubMed Central PMCID: PMC6315778.

Imai C, Iwamoto S, Campana D. Genetic modification of primary natural killer cells overcomes inhibitory signals and induces specific killing of leukemic cells. Blood. 2005 Jul. 1; 106(1):376-83. Epub 2005 Mar. 8. PubMed PMID: 15755898; PubMed Central PMCID: PMC1895123.

Kasinath B S, Mariappan M M, Sataranatarajan K, Lee M J, Feliers D. mRNA translation: unexplored territory in renal science. J Am Soc Nephrol. 2006 December; 17(12):3281-92. Epub 2006 Sep. 7. Review. PubMed PMID: 16959824.

Kebriaei P, et al. Phase I trials using Sleeping Beauty to generate CD19-specific CAR T cells. J Clin Invest. 2016 Sep. 1; 126(9):3363-76. doi: 10.1172/JCI86721. Epub 2016 Aug. 2. PubMed PMID: 27482888; PubMed Central PMCID: PMC5004935.

Khan S H. Genome-Editing Technologies: Concept, Pros, and Cons of Various Genome-Editing Techniques and Bioethical Concerns for Clinical Application. Mol Ther Nucleic Acids. 2019 Jun. 7; 16:326-334. doi: 10.1016/j.omtn.2019.02.027. Epub 2019 Apr. 3. Review. PubMed PMID: 30965277; PubMed Central PMCID: PMC6454098.

Koning F, Lew A M, Maloy W L, Valas R, Coligan J E. The biosynthesis and assembly of T cell receptor alpha- and beta-chains with the CD3 complex. J Immunol. 1988 May 1; 140(9):3126-34. PubMed PMID: 2966207.

Lee A S, Kranzusch P J, Cate J H. eIF3 targets cell-proliferation messenger RNAs for translational activation or repression. Nature. 2015 Jun. 4; 522(7554):111-4. Epub 2015 Apr. 6. PubMed PMID: 25849773; PubMed Central PMCID: PMC4603833.

Lee A S, Kranzusch P J, Doudna J A, Cate J H. eIF3d is an mRNA cap-binding protein that is required for specialized translation initiation. Nature. 2016 Aug. 4; 536(7614):96-9. Epub 2016 Jul. 27. PubMed PMID: 27462815; PubMed Central PMCID: PMC5003174.

Lee Y H, Kim C H. Evolution of chimeric antigen receptor (CAR) T cell therapy: current status and future perspectives. Arch Pharm Res. 2019 Mar. 4. [Epub] Review. PubMed PMID: 30830661.

Liu E, et al. Cord blood NK cells engineered to express IL-15 and a CD19-targeted CAR show long-term persistence and potent antitumor activity. Leukemia. 2018 February; 32(2):520-531. Epub 2017 Jul. 20. PubMed PMID: 28725044; PubMed Central PMCID: PMC6063081.

Liu J J, et al. CasX enzymes comprise a distinct family of RNA-guided genome editors. Nature. 2019 February; 566(7743):218-223. Epub 2019 Feb. 4. Erratum in: Nature. 2019 April; 568(7752):E8-E10. PubMed PMID: 30718774; PubMed Central PMCID: PMC6662743.

Matsumoto M, Oyamada K, Takahashi H, Sato T, Hatakeyama S, Nakayama K I. Large-scale proteomic analysis of tyrosine-phosphorylation induced by T-cell receptor or B-cell receptor activation reveals new signaling pathways. Proteomics. 2009 July; 9(13):3549-63. PubMed PMID: 19609962.

McGinnis S, Madden T L. BLAST: at the core of a powerful and diverse set of sequence analysis tools. Nucleic Acids Res. 2004 Jul. 1; 32(Web Server issue):W20-5. PubMed PMID: 15215342; PubMed Central PMCID: PMC441573.

Miyamoto S, Patel P, Hershey J W. Changes in ribosomal binding activity of eIF3 correlate with increased translation rates during activation of T lymphocytes. J Biol Chem. 2005 Aug. 5; 280(31):28251-64. Epub 2005 Jun. 9. PubMed PMID: 15946946.

Nishimura T, Nakauchi H. Generation of Antigen-Specific T Cells from Human Induced Pluripotent Stem Cells. Methods Mol Biol. 2019; 1899:25-40. doi: 10.1007/978-1-4939-8938-6_3. PubMed PMID: 30649763.

Oei V Y S, et al. Intrinsic Functional Potential of NK-Cell Subsets Constrains Retargeting Driven by Chimeric Antigen Receptors. Cancer Immunol Res. 2018 April; 6(4):467-480.CIR-17-0207. Epub 2018 Feb 19. PubMed PMID: 29459477.

Ohashi P S, et al. Reconstitution of an active surface T3/T-cell antigen receptor by DNA transfer. Nature. 1985 Aug. 15-21; 316(6029):606-9. PubMed PMID: 4033759.

Paucek R D, Baltimore D, Li G. The Cellular Immunotherapy Revolution: Arming the Immune System for Precision Therapy. Trends Immunol. 2019 Mar. 11. pii: 51471-4906(19)30025-0. doi: 10.1016/j.it.2019.02.002. [Epub] Review. PubMed PMID: 30871979.

Rogers S, Wells R, Rechsteiner M Amino acid sequences common to rapidly degraded proteins: the PEST hypothesis. Science. 1986 Oct. 17; 234(4774):364-8 PubMed PMID: 2876518.

Roth T L, et al. Reprogramming human T cell function and specificity with non-viral genome targeting. Nature. 2018 July; 559(7714):405-409. doi: 10.1038/s41586-018-0326-5. Epub 2018 Jul. 11. PubMed PMID: 29995861; PubMed Central PMCID: PMC6239417.

Roth T L, et al. Rapid discovery of synthetic DNA sequences to rewrite endogenous T cell circuits. BioRxiv. 2019. https://doi.org/10.1101/604561.

Sadelain M, Rivière I, Riddell S. Therapeutic T cell engineering. Nature. 2017 May 24; 545(7655):423-431. Review. PubMed PMID: 28541315; PubMed Central PMCID: PMC5632949.

Schumann K, et al.. Generation of knock-in primary human T cells using Cas9 ribonucleoproteins. Proc Natl Acad Sci USA. 2015 Aug. 18; 112(33):10437-42. doi: 10.1073/pnas.1512503112. Epub 2015 Jul. 27. PubMed PMID: 26216948; PubMed Central PMCID: PMC4547290.

Souza-Fonseca-Guimaraes F, Cursons J, Huntington N D. The Emergence of Natural Killer Cells as a Major Target in Cancer Immunotherapy. Trends Immunol. 2019 February; 40(2):142-158. doi: 10.1016/j.it.2018.12.003. Epub 2019 Jan. 10. Review. PubMed PMID: 30639050.

Voon D C, et al. Use of mRNA- and protein-destabilizing elements to develop a highly responsive reporter system. Nucleic Acids Res. 2005 Feb. 16; 33(3):e27. PubMed PMID: 15716309; PubMed Central PMCID: PMC549429.

Zhang Q, Lu W, Liang CL, Chen Y, Liu H, Qiu F, Dai Z. Chimeric Antigen Receptor (CAR) Treg: A Promising Approach to Inducing Immunological Tolerance. Front Immunol. 2018 Oct. 12; 9:2359 eCollection 2018. Review. PubMed PMID: 30369931; PubMed Central PMCID: PMC6194362.

1. A method of modulating engineered immune cell receptor translation using a noncoding sequence element, comprising:
providing an immune cell comprising an engineered cell surface receptor gene encoding a heterologous untranslated region (UTR) comprising one or more eukaryotic initiation factor 3 (eIF3) responsive sites sufficient to modulate translation of the cell surface receptor.

2. The method of claim 1 wherein the heterologous UTR comprises one or more deletion mutations of the one or more eIF3 responsive sites.

3. The method of claim 1 wherein the heterologous UTR comprises the one or more eIF3-responsive sites embedded in a heterologous 3′-UTR sequence.

4. The method of claim 1 wherein the engineered cell surface receptor is a chimeric antigen receptor (CAR) or a T cell receptor (TCR) or a NK cell receptor (NKR).

5. The method of claim 1 wherein the noncoding sequences or responsive sites are in the heterologous 3′ UTR of the gene.

6. The method of claim 1 wherein the heterologous noncoding sequences or responsive sites are human or are Hominidai, Hylobatidae, Cercopithecidae, Perissodactyla, Artiodactyla, Carnivora or Chiroptera in origin.

7. The method of claim 1 in a method of cancer immunotherapy, known as adoptive cell therapy (ACT), or other cell-based therapies using engineered regulatory T cells (engineered Tregs) to treat immune dysfunction such as autoimmunity or organ transplant rejection.

8. The method of claim 1 further comprising the step of introducing the cell into a human host in need of a cell-based therapy.

9. The method of claim 1 further comprising an antecedent step of introducing the gene into the cell, by for examples, using a CRISPR/Cas system comprising, for example a Cas9, Cas12a, CasX, CasY or by using an engineered nucleases such as zinc-finger nucleases, TALENs, MegaTALs or meganucleases.

10(21). A nucleic acid comprising a nucleotide sequence encoding a recombinant polypeptide wherein said nucleic acid further comprises one or more heterologous untranslated elements, optionally wherein:
the nucleic acid is DNA or RNA;
the polypeptide encodes a receptor;
the encoded receptor is a T cell receptor (TCR), chimeric antigen receptor (CAR) or natural killer cell receptor (NKR);
the one or more heterologous untranslated element is/are located in a heterologous untranslated region of the nucleic acid;
a heterologous untranslated element comprises one or more eIF3 responsive elements;
the one or more eIF3 responsive elements are located in the heterologous 3′ untranslated region of the nucleic acid;
the TCR, CAR or NKR is engineered;
the eIF3 responsive element is mammalian;
the eIF3 responsive element is Hominidai, Hylobatidae, Cercopithecidae, Perissodactyla, Artiodactyla, Carnivora or Chiroptera in origin;
the eIF3 responsive element has 70% sequence identity or greater to an eIF3 responsive element that is Hominidai, Hylobatidae, Cercopithecidae, Perissodactyla, Artiodactyla, Carnivora or Chiroptera in origin;
the nucleic acid is incorporated into a vector; and/or
the vector is selected from a plasmid, a particle or a viral vector.

11(34). A cell comprising the nucleic acid of claim 10, optionally wherein:
the nucleic acid is inserted into the genome of the cell or is not inserted into the genome of the cell;
the nucleic acid is inserted into the genome of the cell using a CRISPR/Cas system, zinc finger nucleases, TALENs, MegaTals or a meganuclease;
the cell is a mammalian cell;
the cell is an autologous cell or an allogenic cell;
the cell is selected from a T cell, a B cell, an NK cell or a stem cell;
the T cell is selected from an effector T cell or a regulatory T cell;
the T cell comprises further genomic modifications including insertions and/or deletions;
the T cell wherein insertions and/or deletions result in the knock out of one or more endogenous genes;
the T cell wherein knocked out genes encode polypeptides selected from checkpoint inhibitors, cytokine receptors, endogenous TCRs, and histocompatibility antigens;
the T cell wherein insertions include insertion of a heterologous donor sequence encoding a polypeptide;
the cell is a hematopoietic stem cell;
the stem cell wherein the stem cell comprises further genomic modifications including insertions and/or deletions; and/or the stem cell is incorporated into a composition.

12(10). A recombinant polynucleotide of claim 10, comprising a heterologous untranslated region (UTR) element, said heterologous UTR element comprising one or more eukaryotic initiation factor 3 (eIF3) responsive sites sufficient to modulate the translation of an operably linked sequence encoding a recombinant protein, optionally wherein:
the nucleic acid encoding a recombinant protein, encodes a cell surface receptor, and is operably linked to the eIF3-responsive heterologous UTR element;
the heterologous UTR comprises one or more deletion mutations of the one or more eIF3 responsive sites;
the heterologous UTR comprises the one or more eIF3-responsive sites embedded in a heterologous 3′ -UTR sequence;
the cell surface receptor is a chimeric antigen receptor (CAR);
the heterologous noncoding sequences or responsive sites are in the 3′ UTR of the gene;
the heterologous noncoding sequences or responsive sites are human or are Hominidai, Hylobatidae, Cercopithecidae, Perissodactyla, Artiodactyla, Carnivora or Chiroptera in origin;
the recombinant polynucleotide is incorporated in a vector; and/or the recombinant polynucleotide is incorporated in a cell.

13(19). An immune cell comprising an engineered receptor gene encoding an engineered receptor operably linked to the recombinant polynucleotide of claim 10.

14(20). A cell-based composition adapted and configured for adoptive cell therapy (ACT), or other cell-based therapies using engineered regulatory T cells (engineered Tregs) to treat immune dysfunction such as autoimmunity or organ transplant rejection, and comprising an immune cell comprising an engineered receptor gene encoding an engineered receptor operably linked to the recombinant polynucleotide of claim 10.

15(48). A method for modulating the translation of a polypeptide in a cell using a heterologous non-coding sequence element comprising:
providing a cell comprising a gene encoding a polypeptide wherein said gene comprises one or more heterologous untranslated regions (UTRs), optionally wherein:
the polypeptide is a receptor;
wherein the receptor is selected from a T cell receptor (TCR), chimeric antigen receptor (CAR) or natural killer cell receptor (NKR);
the one or more heterologous untranslated element is/are located in an untranslated region (UTR) of the nucleic acid;
said UTR comprises one or more eIF3 responsive elements;
the one or more eIF3 responsive elements are located in the 3′ UTR of the nucleic acid;
the cell is selected from a T cell, a B cell, an NK cell or a stem cell;
the method or a foregoing the composition is used in cancer immunotherapy for adoptive cell therapy (ACT), or other cell-based therapies to treat immune dysfunction or organ transplant rejection; and/or
further comprising the step of introducing the cell into a human host in need thereof.


SEQUENCE LISTING (21 sequences)

SEQ ID NO: 1 - DNA, 374 bp, Homo sapiens
aaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcatta

SEQ ID NO: 2 - DNA, 135 bp, Homo sapiens
tcacccaggattctcctgtacctgctcccaatctgtgttcctaaaagtgattctcactctgcttctcatctcctacttacatgaatacttctctcttttttctgtttccctgaagattgagctcccaacccccaa

SEQ ID NO: 3 - DNA, 256 bp, Homo sapiens
ggctgactggcatgaggaagctacactcctgaagaaaccaaaggcttacaaaaatgcatctccttggcttctgacttctttgtgattcaagttgacctgtcatagccttgttaaaatggctgctagccaaaccactttttcttcaaagacaacaaacccagctcatcctccagcttgatgggaagacaaaagtcctggggaaggggggtttatgtcctaactgctttgtatgctgttttataaagggatagaagga

SEQ ID NO: 4 - DNA, 444 bp, Homo sapiens
cagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcactctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaataagcaaaataaatttaaaaaaatgaaaagt

SEQ ID NO: 5 - protein, 7 aa, Campylobacter jejuni
NNNVRYM

SEQ ID NO: 6 - DNA, 374 bp, Homo sapiens; identical to SEQ ID NO: 1

SEQ ID NO: 7 - DNA, 135 bp, Homo sapiens; identical to SEQ ID NO: 2

SEQ ID NO: 8 - DNA, 256 bp, Homo sapiens; identical to SEQ ID NO: 3

SEQ ID NO: 9 - DNA, 444 bp, Homo sapiens; identical to SEQ ID NO: 4

SEQ ID NO: 10 - RNA, 20 nt, Homo sapiens
gaacucuccuacccccaagg

SEQ ID NO: 11 - RNA, 20 nt, Homo sapiens
gggaaauuaucucauuaucg

SEQ ID NO: 12 - RNA, 20 nt, Homo sapiens
gugaggaugaagaaugaccu

SEQ ID NO: 13 - RNA, 20 nt, Homo sapiens
uuagccuauuucguacuugg

SEQ ID NO: 14 - DNA, 20 bp, Homo sapiens
tgtcagtgattgggttccga

SEQ ID NO: 15 - DNA, 20 bp, Homo sapiens
cagatctcagctggaccaca

SEQ ID NO: 16 - DNA, 20 bp, Homo sapiens
ctccagatactgcctgagca

SEQ ID NO: 17 - DNA, 20 bp, Homo sapiens
ctatcctgggtccactcgtc

SEQ ID NO: 18 - DNA, 20 bp, Photinus pyralis
tcccgtatgaaggtctgagc

SEQ ID NO: 19 - DNA, 20 bp, Photinus pyralis
tcatccacagggtacaccac

SEQ ID NO: 20 - DNA, 20 bp, Aequorea victoria
tacttctcgatgcgggtgtt

SEQ ID NO: 21 - DNA, 20 bp, Aequorea victoria
tctaccacttcggcacctac